(19) United States
(12) Patent Application Publication
(10) Pub. No.: US 2018/0086749 A1
(43) Pub. Date: Mar. 29, 2018

(54) INHIBITORS OF VIRUSES

(71) Applicants: University of Kansas, Lawrence, KS (US); University of Louisville Research Foundation, Inc., Louisville, KY (US)

(72) Inventors: Jennifer E. Golden, Olathe, KS (US); Donghoon Chung, Louisville, KY (US)

(21) Appl. No.: 15/573,240

(22) PCT Filed: May 10, 2016
(86) PCT No.: PCT/US16/31662

§ 371 Date: Nov. 10, 2017

Related U.S. Application Data
(60) Provisional application No. 62/159,897, filed on May 11, 2015.

(51) Int. Cl. C07D 417/12; C07D 417/04; C07D 277/82

(52) U.S. Cl. CPC C07D 417/12; C07D 277/82; C07D 417/04

(57) ABSTRACT

The present technology is directed to compounds, compositions, and methods to treat a viral infection. The compound is according to Formula I or a solvate and/or pharmaceutically acceptable salt thereof. The technology is especially suited to treat Chikungunya virus, Zika virus, Venezuelan equine encephalitis virus, and/or respiratory syncytial virus infection.

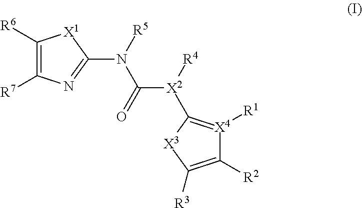

### CROSS-REFERENCE TO RELATED APPLICATIONS

This application claims priority to U.S. Provisional Patent Application No. 62/159,897, filed May 11, 2015, the entire disclosure of which is hereby incorporated by reference in its entirety for any and all purposes.

### GOVERNMENT LICENSE RIGHTS

This invention was made with support from the government under U54 HG005031 awarded by the National Institutes of Health. The government has certain rights in the invention.

### FIELD

The present technology is directed to compounds, compositions, and methods to treat a virus, such as a Flaviviridae virus, Filoviridae virus, Retroviridae virus, Arenaviridae virus, Coronaviridae virus, Orthomyxoviridae virus, Paramyxoviridae virus, Togaviridae virus, or Rhabdovidae virus. The technology is especially suited to treat a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus.

### SUMMARY

The present technology is directed to compounds, compositions, and methods to treat a virus. The compounds and compositions described herein may be used in the treatment or prophylaxis of diseases that include, for example, infections by viruses, such as a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus.

Thus, in an aspect, a compound according to Formula I is provided

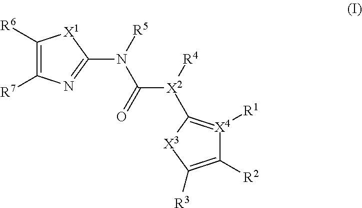

or a solvate and/or pharmaceutically acceptable salt thereof; wherein X¹ is NH, S, or O; X² is N, CH, or C-(unsubstituted alkyl); X³ is S, O, or —C(R⁸)═C(R⁹)—; X⁴ is N or C; R², R³, R⁸ and R⁹ are each independently H, halo, cyano, trifluoromethyl, nitro, pentafluorosulfanyl, or a substituted or unsubstituted alkyl, alkoxy, aryl, aryloxy, alkynyl, cycloalkyl, heterocyclylalkyl, alkanoyl, alkanoyloxy, aryloyl, aryloyloxy, carboxylate, or ester group; provided that when X⁴ is N, then R¹ is absent; R⁴ is H or unsubstituted alkyl; R⁵ is H, or a substituted or unsubstituted alkyl, alkoxy, aryl, heteroaryl, alkynyl, cycloalkyl, heterocyclyl, heterocyclylalkyl, alkanoyl, or aryloyl group; or R⁴ and R⁵ taken together are a substituted or unsubstituted C₂-C₄ alkylenyl group; and R⁶ and R⁷ are each independently H or unsubstituted alkyl, or R⁶ and R⁷ taken together form a substituted or unsubstituted aryl, or R⁶ and R⁷ taken together are a substituted or unsubstituted C₃-C₅ alkylenyl or —(CH₂)ₓ—O—(CH₂)_{y}— group, where x and y are each independently 1, 2, or 3 provided that when x is 3, y is 1, and when y is 3, x is 1.

### BRIEF DESCRIPTION OF THE DRAWINGS

FIG. 1. illustrates the results of testing for cytotoxicity of ML416, a compound of the present technology. The cytotoxicity of ML416 in Vero 76 cells was >50 μM. Cell viability assay was done over the course of four days; 48-hours, 72-hours, and 96-hours exposure. Each data point represents the mean of percent cell viability from triplicates. Dose-Response curve and IC₅₀ were generated using the Four Parameter Logistic Model or Sigmoidal Dose-Response model.

FIG. 2 illustrates the viability, toxicity and apoptosis induced by the treatment of ML416 was measured by using ApoTox-Glo Triplex Assay (Promega). Viability was measured with a cell permeant fluorogenic protease substrate (GF-AFC) and toxicity was measured with fluorogenic cell-impermeant peptide substrate (bis-AAF-R110). Apoptosis was measured by using a luminogenic caspase-3/7 substrate. Each data point represent the mean from triplicates.

FIGS. 3A & 3B illustrate the results when one day-old HEK 293T cells were treated with DMSO, MPA (1 μM) or ML416 (1 μM) in the presence of the denoted supplements for two hours, whereafter the cells were infected with a luciferase-tagged V3526 strain (V3526-luc; FIG. 3A) or luciferase-tagged pseudotype Vesicular stomatitis virus (pVSV-luc; FIG. 3B). After eighteen hours of incubation, the luciferase activity from the infected cells were measured. The values represent the means and their standard deviations of 4 replicates samples as a percentage of the values for DMSO control wells.

FIG. 4 illustrate the results when one-day-old HEK 293T cells were treated with 5 μM of ML416 or DMSO for 8 hours, then infected with mock or pseudotypeVSV-luc at a multiplicity of infection (“MOI”; Plaque forming units (pfu) of virus used for infection/number of cells) of 3. Cells were further incubated for 16 hours in the presence of ML416 or DMSO then the host gene expression level was measured using quantitative real-time RT-PCR.

FIG. 5 illustrates the results where HEK 293T cells were treated with ML416 or DMSO for 18 hours and the cell culture media were subjected to IFN α/β ELISA assay to detect interferons. 125 pg/mL of IFN-α and IFN-β were used as controls.

FIG. 6 illustrates the results where HEK-Blue™ IFN-α/β reporter cells were incubated with 5 mM of ML416 or DMSO for 18 hours and then the expressed secreted alkaline phosphatase (SEAP), which is controlled by IRF9 was measured.

FIG. 7 illustrates the drug induced inhibition of Ebola by overnight pretreatment of a compound of the present technology as well as a 1 hour pretreatment by the same compound of the present technology in comparison with a untreated control HeLa cells.

### DETAILED DESCRIPTION

In various aspects, the present technology provides compounds and methods for treating a viral infection. The compounds provided herein can be formulated into pharmaceutical compositions and medicaments that are useful in the disclosed methods. Also provided is the use of the compounds in preparing pharmaceutical formulations and medicaments, the use of the compounds in treating a viral infection.

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P, and ³⁵S are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolykazaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —C(O)OH group as well as —C(O)O⁻ group. A “protected carboxylate” refers to a —C(O)O-G where G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, N.Y., (3rd Edition, 1999) which can be added or removed using the procedures set forth therein and which is hereby incorporated by reference in its entirety and for any and all purposes as if fully set forth herein.

The term “ester” as used herein refers to —COOR⁷⁰ groups. R⁷⁰ is a substituted or unsubstituted alkyl, cycloalkyl, alkenyl, alkynyl, aryl, aralkyl, heterocyclylalkyl or heterocyclyl group as defined herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while sulfides include —SR⁸⁰ groups, sulfoxides include —S(O)R⁸¹ groups, sulfones include —SO₂R⁸² groups, and sulfonyls include —SO₂OR⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxy’ as used herein can refer to —OH or its ionized form, —O⁻.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “trifluoromethyldiazirido” refers to

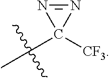 [SMILES: *C1(C(F)(F)F)N=N1]

The term “isocyano” refers to —NC.

The term “isothiocyano” refers to —NCS.

The term “pentafluorosulfanyl” refers to —SF₅.

The phrase “selectively treats” as used herein will be understood by persons of ordinary skill in the art and will vary to some extent depending upon the context in which the phrase is used. If there are uses of the phrase which are not clear to persons of ordinary skill in the art, given the context in which the phrase is used, the phrase at minimum refers to the compounds acting through a viral-specific mechanism of action, resulting in fewer off-target effects because the compounds target the virus and not the host.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g. alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

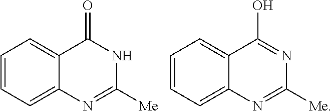

As another example, guanidines may exhibit the following isomeric forms in protic organic solution, also referred to as tautomers of each other:

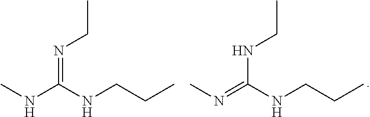

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

Despite the economic and healthcare burden posed by viral infections, current treatments for resulting diseases are limited mostly to prophylactic vaccines. Only a small number of viral diseases (e.g., Human immunodeficiency virus and Hepatitis C virus, HCV) can be treated with virus-specific therapeutics (e.g., sofosbuvir). These agents, so-called direct acting antivirals (DAA), target viral gene products for their activities. In general, DAAs are prone to develop resistant mutants and have a narrow antiviral spectrum. Given the emergence of new viruses and the rapid spread of emerging viral diseases to previously unaffected geographic areas, there is an urgent need for the identification of agents that more efficiently target a broad range of viral diseases, which DAA approaches may not be able to deliver in time.

The alphavirus genus comprises nearly thirty enveloped, positive-sense, single stranded RNA viruses that are typically transmitted by infected insects and are geographically widely distributed. About a third of these are known to contribute to human disease that variably manifests as rash, arthritis, fever, encephalitis, and death in humans. While there currently are no FDA approved therapeutics available to treat any alphavirus infection, a renewed interest to find effective therapeutic leads for development has emerged due to the lack of effective countermeasures for these pathogens and the increased incidence of their prevalence with global climate changes.

For example, Chikungunya virus (CHIKV) is an arthrogenic alphavirus that affects over 60 countries in Asia, Africa, Europe and both American continents. As a NIAID category C priority pathogen, CHIKV is characterized by high fever, rash and debilitating, persistent joint pain that can last from weeks to years. There are no approved vaccines or drugs for Chikungunya virus infection. Current treatments, such and non-steroidal anti-inflammatory agents, only address symptoms of the infection.

In terms of viruses that are not alphaviruses, the Zika virus (ZIKV) has caught recent attention. The Zika virus is spread to people primarily through the bite of an infected Aedes species mosquito. The most common symptoms of a Zika viral infection are fever, rash, joint pain, and conjunctivitis (red eyes). Zika virus infection during pregnancy can cause a serious birth defect called microcephaly, as well as other severe fetal brain defects. Zika virus was first discovered in 1947 and is named after the Zika Forest in Uganda. In 1952, the first human cases of Zika were detected and since then, outbreaks of Zika have been reported in tropical Africa, Southeast Asia, and the Pacific Islands. In May 2015, the Pan American Health Organization (PAHO) issued an alert regarding the first confirmed Zika virus infection in Brazil. On Feb. 1, 2016, the World Health Organization (WHO) declared Zika virus a Public Health Emergency of International Concern (PHEIC). Local transmission has been reported in many other countries and territories. Zika virus will likely continue to spread to new areas. There are no approved vaccines or drugs for Zika virus infection.

The present technology is directed to compounds, compositions, and methods to of treatment or prophylaxis of a virus. The technology is suited to the treatment or prophylaxis of a Flaviviridae virus, Filoviridae virus, Retroviridae virus, Arenaviridae virus, Coronaviridae virus, Orthomyxoviridae virus, Paramyxoviridae virus, Togaviridae virus, or Rhabdovidae virus. The technology is especially suited to the treatment or prophylaxis of a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus. Thus, the present technology is not restricted to the treatment of alphaviruses.

Thus, in an aspect, a compound according to Formula I is provided

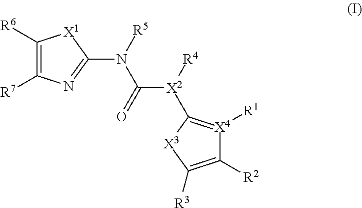

or a solvate and/or pharmaceutically acceptable salt thereof; wherein X¹ is NH, S, or O; X² is N, CH, or C-(unsubstituted alkyl); X³ is S, O, or —C(R⁸)═C(R⁹)—; X⁴ is N or C; R², R³, R⁸ and R⁹ are each independently H, halo, cyano, trifluoromethyl, nitro, pentafluorosulfanyl, or a substituted or unsubstituted alkyl, alkoxy, aryl, aryloxy, alkynyl, cycloalkyl, heterocyclylalkyl, alkanoyl, alkanoyloxy, aryloyl, aryloyloxy, carboxylate, or ester group; provided that when X⁴ is N, then R¹ is absent; R⁴ is H or unsubstituted alkyl; R⁵ is H, or a substituted or unsubstituted alkyl, alkoxy, aryl, heteroaryl, alkynyl, cycloalkyl, heterocyclyl, heterocyclylalkyl, alkanoyl, or aryloyl group; or R⁴ and R⁵ taken together are a substituted or unsubstituted C₂-C₄ alkylenyl group; and R⁶ and R⁷ are each independently H or unsubstituted alkyl, or R⁶ and R⁷ taken together form a substituted or unsubstituted aryl, or R⁶ and R⁷ taken together are a substituted or unsubstituted C₃-C₅ alkylenyl or —(CH₂)ₓ—O—(CH₂)_{y}— group, where x and y are each independently 1, 2, or 3 provided that when x is 3, y is 1, and when y is 3, x is 1.

In any embodiment herein, it may be the compound of Formula I is a compound according to Formula II

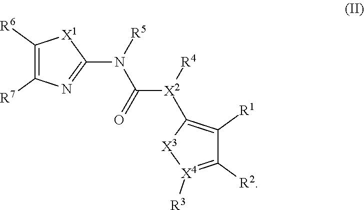

In any embodiment herein, it may be the compound of Formula I or Formula II is a compound according to Formula III

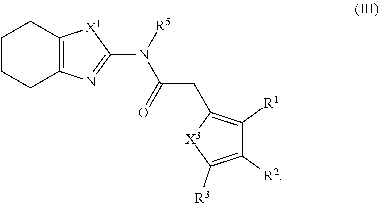

In any embodiment herein, it may be X² is N or CH. In any embodiment herein, R⁴ may be H, or R⁴ and R⁵ together are a substituted or unsubstituted C₂-C₄ alkylenyl group. In any embodiment herein, it may be R⁶ and R⁷ taken together form a substituted or unsubstituted aryl or R⁶ and R⁷ taken together are a substituted or unsubstituted C₃-C₅ alkylenyl. In any embodiment herein, it may be R⁶ and R⁷ taken together form a substituted or unsubstituted phenyl or R⁶ and R⁷ taken together are a substituted or unsubstituted C₃-C₅ alkylenyl. In any embodiment herein, R⁵ may be a substituted or unsubstituted alkyl, a substituted or unsubstituted phenyl, or an unsubstituted heterocyclyl group; R⁵ may be a substituted or unsubstituted alkyl, a substituted or unsubstituted phenyl, or an unsubstituted saturated heterocyclyl group. In any of the embodiments herein, it may be that X³ is S or O. In any of the embodiments herein, it may be that X¹ is S.

In any of the embodiments herein, it may be that when X³ is —C(R⁸)═C(R⁹)—, then R³ is not trifluoromethyl and is not alkoxy. In any embodiment herein, it may be that when X³ is S, then R¹, R², and R³ each are not trifluoromethyl. In any of the embodiments herein, it may be that when X¹ is S; X² is CH; X³ is S; R², R³, R⁴ and R⁵ are each independently H; and R⁶ and R⁷ taken together form a unsubstituted cyclohexyl group; then R¹ is not H. In any of the embodiments herein, it may be that when X¹ is S; X² is CH; X³ is S; R¹, R², R³, and R⁴ are each independently H; and R⁶ and R⁷ taken together form a unsubstituted cyclohexyl group; then R⁵ is not phenyl.

In any of the above embodiments, it may be the ability to inhibit a cytopathic effect of a virus (such as a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus) is realized. For example, ML416 (see Scheme 1) inhibits a CHIKV-induced cytopathic effect in the nanomolar range without showing significant cytotoxicity (>40 μM, selectivity index [EC₅₀/CC₅₀]>140) while also reducing CHIKV viral titer in vitro (4 log) at a 5 μM compound concentration. As discussed in the working examples, this compound exhibited antiviral activity for Zika virus, Venezuelan equine encephalitis virus (VEEV), CHIKV, and respiratory syncytial virus (RSV), among others as described in the working examples.

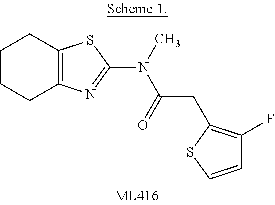 [SMILES: CN(C(=O)Cc1sccc1F)c1nc2c(s1)CCCC2]

The IUPAC name of the ML416 is 2-(3-fluorothiophen-2-yl)-N-methyl-N-(4,5,6,7-tetrahydro-1,3-benzothiazol-2-yl)acetamide.

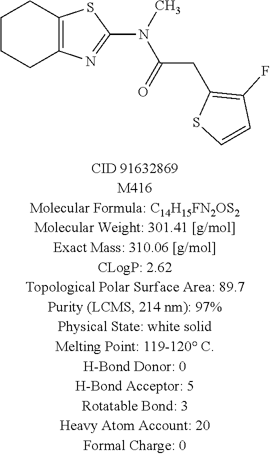 [SMILES: CN(C(=O)Cc1sccc1F)c1nc2c(s1)CCCC2]

In an aspect of the present technology, a composition is provided that includes any one of the embodiments of compounds of Formulas I-III and a pharmaceutically acceptable carrier. In a related aspect, a pharmaceutical composition is provided, the pharmaceutical composition including an effective amount of the compound of any one of the aspects and embodiments of compounds of Formulas I-III for treating a condition; and where the condition is a viral infection. In a further related aspect, a method is provided that includes administering an effective amount of a compound of any one of the aspects and embodiments of compounds of Formulas I-III or administering a pharmaceutical composition comprising an effective amount of a compound of any one of the aspects and embodiments of compounds of Formulas I-III to a subject suffering from a viral infection. The viral infection of any aspect or embodiment herein may include a Flaviviridae virus, Filoviridae virus, Retroviridae virus, Arenaviridae virus, Coronaviridae virus, Orthomyxoviridae virus, Paramyxoviridae virus, Togaviridae virus, or Rhabdovidae virus. The viral infection of any aspect or embodiment herein may include a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus. In the method, risk of infection by and/or transmission of the virus by the subject is decreased.

“Effective amount” refers to the amount of a compound or composition required to produce a desired effect, where an “antiviral effective amount” refers to the amount of a compound or composition required to produce a desired effect in regard to a viral infection. One example of an effective amount includes amounts or dosages that yield acceptable toxicity and bioavailability levels for therapeutic (pharmaceutical) use including, but not limited to, the treatment or prophylaxis of a viral infection (e.g., a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus). Another example of an effective amount includes amounts or dosages that are capable of reducing symptoms associated with a viral infection, such as, for example, fever, headache, and/or encephalitis. As used herein, a “subject” or “patient” is a mammal, such as a cat, dog, rodent or primate. Typically the subject is a human, and, preferably, a human suffering from or suspected of suffering from viral infection, such as a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus. The term “subject” and “patient” can be used interchangeably.

Generally, “prophylactic” or “prophylaxis” relates to a reduction in the likelihood of the patient developing a disorder, such as any one or more of the above-mentioned viral infections, or proceeding to a diagnosis state for the disorder. For example, the compounds of the present technology can be used prophylacticly as a measure designed to preserve health and prevent the spread or maturation of disease in a patient. It is also appreciated that the various modes of treatment or prevention of a disease such as an viral infection can mean “substantial” treatment or prevention, which includes total but also less than total treatment or prevention, and in which some biologically or medically relevant result is achieved. Furthermore, treatment or treating as well as alleviating can refer to therapeutic treatment and prophylactic or preventative measures in which the object is to prevent, slow down (lessen) a disease state, condition or malady. For example, a subject can be successfully treated for an viral infection if, after receiving through administration an effective or therapeutic amount of one or more compounds described herein, the subject shows observable and/or measurable reduction in or absence of one or more signs and symptoms of the particular disease. The present technology also provides for methods of administering one or more compounds of the present technology to a patient in an effective amount for the treatment or prophylaxis of a viral infection such as, for example, an infection caused by one or more of a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus.

Thus, the instant present technology provides pharmaceutical compositions and medicaments comprising any of the compounds disclosed herein (e.g., compounds of Formulas I-III) and a pharmaceutically acceptable carrier or one or more excipients or fillers (collectively, such carriers, excipients, fillers, etc., will be referred to as “pharmaceutically acceptable carriers” unless a more specific term is used). The compositions may be used in the methods and treatments described herein. Such compositions and medicaments include a therapeutically effective amount of any compound as described herein, including but not limited to a compound of Formulas I-III, for treating one or more of the herein-described conditions. The pharmaceutical composition may be packaged in unit dosage form. For example, the unit dosage form is effective in treating a viral infection (e.g., caused by a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus) by reducing symptoms associated with the infection when administered to a subject in need thereof.

The pharmaceutical compositions and medicaments may be prepared by mixing one or more compounds of the present technology, pharmaceutically acceptable salts thereof, stereoisomers thereof, tautomers thereof, or solvates thereof, with pharmaceutically acceptable carriers, excipients, binders, diluents or the like to prevent and treat disorders associated with viral infections, such as by a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus. The compounds and compositions described herein may be used to prepare formulations and medicaments that prevent or treat a variety of disorders associated with such viral infections. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. An isotonic solution will be understood as isotonic with the subject. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until, for example, progression of the disease state and/or elevated plasma white blood cell count is decreased or stopped. The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition being treated and the pharmacological activity of the compound being used. The determination of optimum dosages for a particular patient is well known to those skilled in the art.

Various assays and model systems can be readily employed to determine the therapeutic effectiveness of the treatment according to the present technology.

Effectiveness of the compositions and methods of the present technology may also be demonstrated by a decrease in the symptoms of a viral infection, such as, for example, fever, headache, and/or encephalitis

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

The compounds of the present technology can also be administered to a patient along with other conventional therapeutic agents that may be useful in the treatment or prophylaxis of viral infections. Thus, a pharmaceutical composition of the present technology may further include an antiviral different than the compounds of Formulas I-III. For example, the pharmaceutical composition may further include an effective amount of an antiviral. The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology can also comprise administering, either sequentially or in combination with one or more compounds of the present technology, a conventional therapeutic agent in an amount that can potentially or synergistically be effective for the treatment of viral infections.

In one aspect, a compound of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

A compound of the present technology can also be modified, for example, by the covalent attachment of an organic moiety or conjugate to improve pharmacokinetic properties, toxicity or bioavailability (e.g., increased in vivo half-life). The conjugate can be a linear or branched hydrophilic polymeric group, fatty acid group or fatty acid ester group. A polymeric group can comprise a molecular weight that can be adjusted by one of ordinary skill in the art to improve, for example, pharmacokinetic properties, toxicity or bioavailability. Exemplary conjugates can include a polyalkane glycol (e.g., polyethylene glycol (PEG), polypropylene glycol (PPG)), carbohydrate polymer, amino acid polymer or polyvinyl pyrolidone and a fatty acid or fatty acid ester group, each of which can independently comprise from about eight to about seventy carbon atoms. Conjugates for use with a compound of the present technology can also serve as linkers to, for example, any suitable substituents or groups, radiolabels (marker or tags), halogens, proteins, enzymes, polypeptides, other therapeutic agents (for example, a pharmaceutical or drug), nucleosides, dyes, oligonucleotides, lipids, phospholipids and/or liposomes. In one aspect, conjugates can include polyethylene amine (PEI), polyglycine, hybrids of PEI and polyglycine, polyethylene glycol (PEG) or methoxypolyethylene glycol (mPEG). A conjugate can also link a compound of the present technology to, for example, a label (fluorescent or luminescent) or marker (radionuclide, radioisotope and/or isotope) to comprise a probe of the present technology. Conjugates for use with a compound of the present technology can, in one aspect, improve in vivo half-life. Other exemplary conjugates for use with a compound of the present technology as well as applications thereof and related techniques include those generally described by U.S. Pat. No. 5,672,662, which is hereby incorporated by reference herein.

In another aspect, the present technology provides methods of identifying a target of interest including contacting the target of interest with a detectable or imaging effective quantity of a labeled compound of the present technology. A detectable or imaging effective quantity is a quantity of a labeled compound of the present technology necessary to be detected by the detection method chosen. For example, a detectable quantity can be an administered amount sufficient to enable detection of binding of the labeled compound to a target of interest including, but not limited to, a viral protein, viral DNA, or viral RNA. Suitable labels are known by those skilled in the art and can include, for example, radioisotopes, radionuclides, isotopes, fluorescent groups, biotin (in conjunction with streptavidin complexation), and chemoluminescent groups. Upon binding of the labeled compound to the target of interest, the target may be isolated, purified and further characterized such as by determining the amino acid sequence of a protein to which the labeled compound of the present technology is bound.

The terms “associated” and/or “binding” can mean a chemical or physical interaction, for example, between a compound of the present technology and a target of interest. Examples of associations or interactions include covalent bonds, ionic bonds, hydrophilic-hydrophilic interactions, hydrophobic-hydrophobic interactions and complexes. Associated can also refer generally to “binding” or “affinity” as each can be used to describe various chemical or physical interactions. Measuring binding or affinity is also routine to those skilled in the art. For example, compounds of the present technology can bind to or interact with a target of interest or precursors, portions, fragments and peptides thereof and/or their deposits.

The examples herein are provided to illustrate advantages of the present technology and to further assist a person of ordinary skill in the art with preparing or using the compounds of the present technology or salts, pharmaceutical compositions, derivatives, metabolites, prodrugs, racemic mixtures or tautomeric forms thereof. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### Examples

General Experimental and Analytical Details:

¹H and ¹³C NMR spectra were recorded on a Bruker AM 400 spectrometer (operating at 400 and 101 MHz respectively) or a Bruker AVIII spectrometer (operating at 500 and 126 MHz respectively) in CDCl₃ with 0.05% TMS as an internal standard. The chemical shifts (δ) reported are given in parts per million (ppm) and the coupling constants (J) are in Hertz (Hz). The spin multiplicities are reported as s=singlet, br s=broad singlet, d=doublet, t=triplet, q=quartet, p=pentuplet, dd=doublet of doublet, td=triplet of doublet and m=multiplet. The LC-MS analysis was performed on an Agilent 1200 RRL chromatograph with photodiode array UV detection and an Agilent 6224 TOF mass spectrometer. The chromatographic method utilized the following parameters: a Waters Acquity BEH C-18 2.1 mm×50 mm, 1.7 μm column; UV detection wavelength=214 nm; flow rate=0.4 mL/min; gradient=5-100% MeCN over 3 min with a hold of 0.8 min at 100% MeCN; the aqueous mobile phase contained 0.15% NH₄OH. The mass spectrometer utilized the following parameters: an Agilent multimode source that simultaneously acquires ESI+/APCI+; a reference mass solution consisting of purine and hexakis(1H,1H,3H-tetrafluoropropoxy) phosphazine; and a makeup solvent of 90:10:0.1 MeOH/H₂O/HCO₂H which was introduced to the LC flow prior to the source to assist ionization. Melting points were determined on a Stanford Research Systems OptiMelt apparatus. Flash chromatography separations were carried out using a Teledyne Isco CombiFlash Rf 200 purification system with silica gel columns.

Exemplary Synthesis of a Compound of the Present Technology

ML416 was prepared by the route depicted in Scheme 2. An Arndt-Eistert homologation was implemented with 3-fluorothiophene-2-carboxylic acid (1) to generate 2-(3-fluorothiophen-2-yl)acetic acid (3). Coupling of the free carboxylic acid of 3 with the methylated amine of a commercially available tetrahydrobenzothiazolamine afforded desired compound 4 (i.e., ML416).

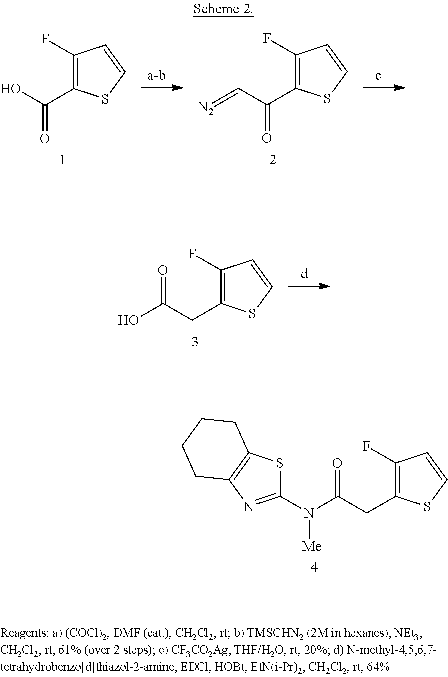

Detailed protocols used for the assembly of ML416 are as follows:

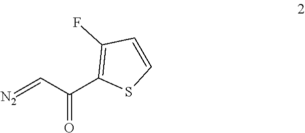

### 2-Diazo-1-(3-fluorothiophen-2-yl)ethanone

Using a large vial, 3-fluorothiophene-2-carboxylic acid (292 mg, 2.0 mmol) was dissolved in dry CH₂Cl₂ (10 mL) under Ar. Dry DMF (5 drops) was added to the solution, followed by dropwise addition of (COCl)₂ (0.19 mL, 2.2 mmol). The mixture was stirred at rt for 30 min. Triethylamine (0.56 mL, 4.0 mmol) was added to the mixture, followed by (dropwise) a solution of trimethylsilyldiazomethane (2.0 M in hexanes, 4 mL, 8.0 mmol). The mixture was stirred at rt for 17 hours. The reaction mixture was slowly quenched with 10% aq. citrate (12 mL). The layers were separated and the aq. layer was extracted with CH₂Cl₂ (20 mL). The combined org. layers were dried with Na₂SO₄. The product was purified by flash chromatography (0-25% EtOAc/hexanes, 24 g column) to give the title compound (208 mg, 61%) as a pale-yellow solid. ¹H NMR (400 MHz, CDCl₃) δ 7.49 (dd, J=5.5, 3.8 Hz, 1H), 6.85 (d, J=5.5 Hz, 1H), 5.99 (s, 1H).

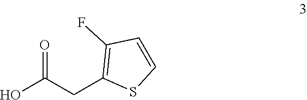

### 2-(3-Fluorothiophen-2-yl)acetic acid

Silver trifluoroacetate (281 mg, 1.27 mmol) was added to a stirring mixture of 2-diazo-1-(3-fluorothiophen-2-yl)ethanone (206 mg, 1.21 mmol) in THF (16 mL) and water (7 mL). The reaction mixture was stirred at rt for 65 hours. The product was extracted with CH₂Cl₂ (2×50 mL), dried with Na₂SO₄ and purified by flash chromatography (isocratic, 100% CH₂Cl₂, 12 g column) to give the title compound (39 mg, 20%) as a white solid. ¹H NMR (400 MHz, CDCl₃) δ 7.13 (dd, J=5.6, 4.0 Hz, 1H), 6.78 (dd, J=5.6, 0.9 Hz, 1H), 3.81 (d, J=1.0 Hz, 2H).

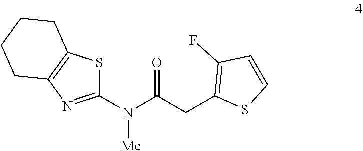 [SMILES: CN(C(=O)Cc1sccc1F)c1nc2c(s1)CCCC2]

### 2-(3-Fluorothiophen-2-yl)-N-methyl-N-(4,5,6,7-tetrahydrobenzo[d]thiazol-2-yl)acetamide

A mixture of N-methyl-4,5,6,7-tetrahydrobenzo[d]thiazol-2-amine (50 mg, 0.30 mmol), N,N-diisopropylethylamine (0.08 mL, 0.46 mmol), HOBt (40 mg, 0.30 mmol), EDCI (57 mg, 0.30 mmol) and 2-(3-fluorothiophen-2-yl)acetic acid (38 mg, 0.24 mmol) in CH₂Cl₂ (5 mL) was stirred at rt for 17 hours. The mixture was diluted with ethyl ether (15 mL), washed successively with 1 M aq. HCl (2×12 mL) and saturated aq. NaHCO₃ (2×12 mL), and dried with Na₂SO₄. The product was purified by flash chromatography (0-35% EtOAc/hexanes, 12 g column) to give the title compound (47 mg, 64%) as a white solid, mp 119-120° C. ¹H NMR (500 MHz, CDCl₃) δ 7.14 (t, J=4.8 Hz, 1H), 6.78 (d, J=5.7 Hz, 1H), 4.07 (s, 2H), 3.75 (s, 3H), 2.74-2.63 (m, 4H), 1.91-1.79 (m, 4H). ¹³C NMR (126 MHz, CDCl₃) δ 168.0, 156.8, 156.5, 154.4, 143.8, 124.3, 123.7, 123.6, 116.8, 116.5, 112.3, 112.1, 34.8, 31.5, 26.6, 23.3, 23.2, 22.7. LC-MS: t_{R}=1.96 min, purity=97%. HRMS (m/z): calcd for C₁₄H₁₆FN₂OS₂ (M+H)⁺ 311.0683; found 311.0640.

Assay Conditions

Cells and Viruses:

Vero 76 (ATCC CRL-1587), BHK (ATCC CCL-10), HEp-2 (ATCC CCL-23), Neuro 2A (ATCC CCL-131), SH-SYSY (ATCC CRL-2266), MRC-5 (ATCC CCL-171) and HEK 293T (ATCC CRL-3216) were obtained from ATCC and maintained in Minimum Essential Medium with Earl's modification (MEM-E) containing 10% fetal bovine serum (FBS) and 1× GlutaMAX (Gibco 35050-061) at 37° C. with 5% CO₂. MDCK (Sigma-Aldrich 84121903), RD (ATCC CCL-136) and NIH 3T3 (ATCC CRL-1658) were maintained in Dulbecco's modified Eagle's medium (DMEM) with 10% FBS and 2 mM L-glutamine at 37° C. with 5% CO₂. TZM-bl cells were obtained through the NIH AIDS Research and Reference Reagent Program from John C. Kappes, Xiaoyun Wu, and Tranzyme, Inc. The TZM-bl indicator cell line, used for infectivity assays of HIV-1, is a genetically engineered HeLa cell clone expressing CD4, CXCR4, CCR5, and Tat-responsive firefly luciferase and Escherichia coli β-galactosidase under the control of an HIV-1 long terminal repeat. TZM-bl cells were cultivated in DMEM (containing 4.5 g/liter glucose, L-glutamine, and sodium pyruvate) medium with 10% fetal calf serum, 50 IU/ml penicillin, and 50 μg/ml streptomycin at 37° C. with 5% CO₂.

VEEV TC-83 (lyophilized vaccine from USAMRIID) and V3526 were amplified in and titrated in BHK-21cells. V3526-luc was rescued from the BHK cells transfected a full-length viral RNA derived from pV3526-luc as described in Bernard K A, Klimstra W B, Johnston R E. 2000. Mutations in the E2 glycoprotein of Venezuelan equine encephalitis virus confer heparan sulfate interaction, low morbidity, and rapid clearance from blood of mice. Virology 276:93-103. VEEV TrD (gift from Dr. R. Tesh, World Reference Center for Emerging Viruses and Arboviruses), herpes simplex virus type-2 (HSV-2, gift from Dr. Steinbach, University of Louisville) were grown in Vero 76 cells that were maintained in Dulbecco's-modified essential media (DMEM) with 10% FBS. Lymphocytic Choriomeningitis Virus ARM strain (LCMV-ARM) was a gift from Dr. Lukashevich (University of Louisville). Chikungunya virus S-27 (BEIR NR-13220), West equine encephalitis virus California (WEEV California, ATCC VR-70), Japanese Encephalitis virus SA14 (BEIR NR-2335), West Nile virus NY-99 (gift from Dr. R. Tesh, World Reference Center for Emerging Viruses and Arboviruses), Yellow Fever virus 17D (BEIR VR-1506) were grown in Vero76 cells in a virus infection media (MEM-E with 10% FBS, 1× GlutaMAX and 25 mM of HEPES pH 7.3). Human enterovirus D71 (strain MP4, BEIR NR-472) and human encephalomyocarditis virus MM (BEIR NR-19846) were amplified in RD cells maintained in DMEM with 10% FBS.

To generate wild-type HIV-1 virus HEK293T cells were plated at a density of 6×10⁶ cells/100 mm culture dish 24 hours prior to transfection. Cells were transfected with 10 μg of wild-type HIV-1 proviral clone, pNL4.3, using linear polyethylenimine (PEI; 25 kDa; Polysciences, Inc.), as described in Durocher Y, Perret S, Kamen A. 2002. High-level and high-throughput recombinant protein production by transient transfection of suspension-growing human 293-EBNA1 cells. Nucleic acids research:E9. Culture supernatants were collected 48 hours after transfection and cellular debris was removed by filtration through a 0.45 μm filter. Viral p24 was quantitated using standard p24 ELISA.

Dose-Response Studies

EC₅₀ and CC₅₀ were evaluated in a dose-response format starting from 50 μM by a 2-fold dilution, triplicates for each, in a 96-well format. For a CPE-based assay, cells were seeded in white well plates at a cell density of 12,000 cells per well in a volume of 45 microliters and incubated in an actively humidified incubator with 5.0% CO₂ at 37° C. and 95% humidity for 18 hours. Test compounds diluted in thirty microliters of cell culture media was added to each well. After a two-hours incubation at 37° C. with a 5% CO₂, a 600 pfu of virus (or cell culture media for cytotoxicity assay) was added to the wells in a volume of fifteen microliters then incubated two days in an actively humidified incubator with 5.0% CO₂ at 37° C. and 95% humidity. Cell viability was measured with 90 microliter per well of CellTiter-Glo™ reagent (Promega). Vero 76 cells were used for alphaviruses and RD cells were used for HEV and EMCV assays. For a luciferase-tagged virus assay (i.e., V3526-luc and pVSV-luc), an optimized amount of virus and cell numbers were used for each cell line tested. For HEK 293T, Neuro 2A and SH-SY5Y cells, 24,000 cells and 2,400 TCID₅₀ units of virus per well was used. For Vero 76 and BHK cells, 12,000 cells and 1200 TCID₅₀ units of virus per well was used. For NIH3T3, 24,000 cells and 20,000 TCID₅₀ units of virus per well was used. After an 18-hours incubation with virus, the plates were developed with Bright-Glo™ reagent (Promega) and the luciferase activity was measured as a readout for the virus replication.

For measurement of inhibition of Ebolavirus infection, a recombinant Ebolavirus with a green fluorescent protein (GFP) gene inserted into the genome (Ebola-eGFP virus, a kind gift of Dr. Heinz Feldmann) was used. See Towner J S, Paragas J, Dover JE, Gupta M, Goldsmith C S, Huggins J W, Nichol S T. 2005. Generation of eGFP expressing recombinant Zaire ebolavirus for analysis of early pathogenesis events and high-throughput antiviral drug screening. Virology doi:10.1016/j.virol.2004.10.048:20-27. The virus stock was generated by infecting Vero E6 cells with Ebola-eGFP virus followed by pelleting the culture supernatant through a 20% sucrose cushion. HeLa cells were pretreated for 2 hour with two fold-dilutions of 10 to 0.005 μM of compound and incubated with virus for 24 hours in the presence of the compound. Fixed cells were imaged by a fluorescent microscope. Total and infected cells were counted by Cell Profiler image analysis software (Broad Institute, MIT, Boston, Mass.), detecting nuclei stained with DAPI and virus encoded GFP expression. See Leung D W, Borek D, Luthra P, Binning J M, Anantpadma M, Liu G, Harvey I B, Su Z, Endlich-Frazier A, Pan J, Shabman R S, Chiu W, Davey R A, Otwinowski Z, Basler C F, Amarasinghe G K. 2015. An Intrinsically Disordered Peptide from Ebola Virus VP35 Controls Viral RNA Synthesis by Modulating Nucleoprotein-RNA Interactions. Cell reports doi:10.1016/j.celrep.2015.03.034:376-389. This work was performed in a biosafety level 4 (BSL4) laboratory at Texas Biomedical Research Institute.

Titer Reduction Assay

To measure virus titer reduction, 12-well plates with 100,000 Vero 76 cells grown overnight were pre-treated with compound diluted in virus infection medium at 37° C. with 5% CO₂ for 8 hours, unless denoted. For virus adsorption, cell plates were incubated on ice for a 15 min, then the cell culture supernatant was removed. Virus diluted at an MOI of 0.05 (or 3 for a time of addition study) in 250 microliter of virus infection medium was added to the cell and the virus was allowed to absorb to the cells on ice for one hour. The unabsorbed virus was washed with 1 mL of PBS once and the wells were replenished with virus infection medium with 5 μM of ML416 or DMSO (0.25% vol/vol). The progeny virus was harvested after 26 hours for VEEV, CHIKV, WEEV, HSV-2 or Influenza virus; 48 hours for JEV, YFV17D or WNV; 72 hours for RSV or LCMV virus. The progeny viruses in the supernatants were enumerated by using either virus infection center assay or TCID50 assays. Virus infection center assay was done with Vero76 cells grown confluent in 24-well plates. The cells were infected with 167 μL of the serially diluted virus samples for one hour at 37° C. with 5% CO₂. Wells were washed with PBS and replenished with virus infection medium with 0.75% methylcellulose. Three or four days after virus infection, virus infection centers were visualized with crystal violet staining (0.2% crystal violet, 4% paraformaldehyde, and 10% ethanol). For influenza virus and HSV-2, a TCID₅₀ assay used for the titration.

HIV-1 Viral Infectivity Assay

TZM-bl indicator cells were plated at a density of 10,000 cells/well in a 96 well culture plate 24 hours prior to HIV-1 infection and incubated at 37° C. (5% CO₂). On the day of infection, the culture medium was removed, and the cells were inoculated in triplicate with 100 μl of 2-fold serial dilutions of viral supernatants in culture medium containing 20 μg/ml DEAE-dextran and incubated at 37° C. (5% CO₂). At the time of infection DMSO as a control or 10 μM ML461 were added to test effect of the compound on HIV-1 infection. After a 24 hours of incubation, culture medium was removed from each well and replaced with 100 μl of Britelite Plus luciferase assay substrate (PerkinElmer). Following 5 min of incubation at room temperature, 70 μl of each cell lysate was transferred to a 96-well OptiPlate 96 (PerkinElmer) and luminescence was measured in a VICTOR X2 Multilabel Reader (PerkinElmer). Relative infectivity was calculated by plotting luciferase activity of viral particle with treated DMSO as 100%.

Microarray.

HEp-2 cells were treated for 18 hours either with 5 μM of CID:70698683 or with DMSO for the control in cell culture medium. 100 ng total RNA was amplified and labeled following the Affymetrix (Santa Clara, Calif.) standard protocol for their 3′IVT Plus Labeling Kit, followed by hybridization to Affymetrix' Primeview® Human Gene Expression arrays. The arrays were processed following the manufacturer recommended wash and stain protocol on an Affymetrix FS-450 fluidics station and scanned on an Affymetrix GeneChip® 7G scanner using Command Console 4.0. The resulting .cel files were imported into Partek Genomics Suite 6.6 and transcripts were normalized and summarized using RMA as normalization and background correction method. A 1-way ANOVA was set up to compare the treatment of 5 μM of CID:70698683 to the control. False Discovery Rate (FDR) was chosen as multiple test correction for the resulting p-values.

RealTime PCR

Total RNAs from cells in a 12-well plate were isolated with RNAzol® RT (Molecular Research Center, Inc) reagent as per the manufacturer's protocol and were dissolved in 50 μL of The RNA storage solution (Life Technologies). One microgram of RNA samples were subjected to a cDNA synthesis with Maxima™ H Minus Reverse Transcriptase (Life Technologies), random hexamers, and oligo-dT by following the manufacturer's protocol. For quantitation of gene expression, we used a real-time PCR with 2(−Delta Delta C(T)) method in a total of twenty microliters per well with 2 μL of 2-fold diluted cDNA mixture in a multiplex mode in conjunction with TaqMan chemistry. Information on the primers and probes is provided in Table 1.

TABLE 1
Real-time qPCR primer/probes

| Target gene | Assay I.D. | Company |
|---|---|---|
| OASL | Hs.PT.58.39388489 | IDTDNA |
| IFNB1 | Hs.PT.58.39481063.g | IDTDNA |
| DDX58 | Hs.PT.58.1417383 | IDTDNA |
| STAT1 | Hs.PT.58.15049687 | IDTDNA |
| TRADD | Hs.PT.58.2433448.g | IDTDNA |
| IFIH1 | Hs.PT.58.28238505 | IDTDNA |
| MYD88 | Hs01573837_g1 | Life Technologies |
| IRF3 | Hs01547283_m1 | Life Technologies |
| IRF7 | Hs01014809_g1 | Life Technologies |
| IFIT1 | Hs03027069_s1 | Life Technologies |
| OAS1 | Hs00973637_m1 | Life Technologies |
| OAS2 | Hs00942643_m1 | Life Technologies |
| IRF1 | Hs00971960_m1 | Life Technologies |
| 18S | 4319413E | Life Technologies |
| GAPDH | 4326317E | Life Technologies |

The quantitation of viral genome was done as described in Chung D H, Jonsson C B, Tower N A, Chu Y K, Sahin E, Golden J E, Noah J W, Schroeder C E, Sotsky J B, Sosa M I, Cramer D E, McKellip S N, Rasmussen L, White E L, Schmaljohn C S, Julander J G, Smith J M, Filone C M, Connor J H, Sakurai Y, Davey R A. 2014. Discovery of a novel compound with anti-venezuelan equine encephalitis virus activity that targets the nonstructural protein 2. PLOS Pathogens doi:10.1371/journal.ppat.1004213:e1004213. 18S rRNA (Cat. No. 4319413E, Life Technologies) and human GAPDH (Cat. No. 4326317E Life Technologies) were used as the endogenous controls to quantitate the relative viral and human gene RNA copy numbers, respectively. Three biological replicates along with two technical replicates were used for the quantitation.

Interferon Assays

Interferon-α and -β in the cell culture supernatant were detected by using LumiKine™ Xpress hIFN-α and LumiKine™ hIFN-β kits (InvivoGen) as per the manufacturer's protocol. One-day-old HEK 293T cells cultured in a 12-well plate were treated with 5 μM of ML416 or DMSO in virus infection medium for 18 hours and the cell culture supernatants were harvested and cleared by centrifugation at 3000×g for 10 min. For each treatment, six replicates were used per group and three technical replicates were used for the controls, HEK293-expressed human IFN-α2 and CHO-expressed human IFN-β.

HEK-Blue™ IFN-α/β cell reporter assay (InvivoGen) was done followed by the manufacturer's protocol. One-day-old HEK-Blue™ IFN-α/0 cells plated in a 96-well plate were treated with two fold-dilutions of 50 to 0.4 μM of ML416 or DMSO for 24 hours. The expression of SEAP under the control of ISRE9, which is activated by Type 1 interferons, was measured by the absorbance at 620 nm after 20 min incubation with 100 μL of SEAP substrate. Three replicates were used for each data points.

Metabolomics Analysis

Metabolites were extracted from cell samples using a solvent mixture of acetonitrile, water, and chloroform (2:1.5:1). The metabolite extract from each sample was then derivatized using N-methyl-N-(trimethylsilyl) trifluoroacetamide (MSTFA). All derivatized samples were further analyzed on a LECO Pegasus 4D GC×GC-TOF MS instrument (St. Joseph, Mich.). The instrument data were first processed using LECO's instrument control software ChromaTOF for peak picking and tentative metabolite identification. MetPP software was used for retention index matching, peak merging, peak list alignment, normalization, and statistical significance test. See Wei X, Shi X, Koo I, Kim S, Schmidt R H, Arteel G E, Watson W H, McClain C, Zhang X. 2013. MetPP: a computational platform for comprehensive two-dimensional gas chromatography time-of-flight mass spectrometry-based metabolomics. Bioinformatics (Oxford, England) doi:10.1093/bioinformatics/btt275:1786-1792. The abundance test was performed using pairwise two-tail t-test with sample permutation, to standardize the abundance variation of each metabolite between sample groups. The presence-absence test was performed using Fisher's exact test.

CYP450 Inhibition Profiling—

Inhibition of drug-metabolizing CYP450s—Evaluation of the compound as inhibitor of the activity of CYP3A4, CYP2C9, and CYP1A2 was assessed using the P450-Glo™ Assay kits (Promega) in the presence of human liver microsomes (XenoTech). NADPH, a required cofactor for CYP450 metabolism, was provided by the NADPH Regenerating System, Solutions A (BD Biosciences) and B (BD Biosciences). Compound stock solution was initially prepared in 100% DMSO and subsequently diluted in acetonitrile for the assay. The pH of the reactions was maintained at 7.4 with potassium phosphate buffer (BD Biosciences). The reaction was started after adding NADPH and the corresponding luciferin substrate to the reaction plate containing microsomes and compound. The reaction was incubated for 30 minutes at 37° C. with shaking. Luciferin Detection Reagent was added after the incubation to stop the reaction and initiate a stable glow-type luminescent signal. The luminescence of the metabolite formed, Luciferin, was recorded with the Infinite M200 (Tecan US). All reactions were run in duplicate, except negative controls (no NADPH) which were performed as single reactions. The background luminescence obtained from the negative control reactions was substracted from the luminescence values obtained for the positive reaction duplicates. The percent of inhibition was calculated by the formula: 100−[RLU at 10 μM/RLU at 0 μM*100].

CYP450 Inhibition Profiling—Inhibition of Drug-Metabolizing CYP2C19—

Evaluation of the compound as inhibitor of the activity of CYP2 C19 was assessed using the P450-Glo™ Assay kit (Promega) in the presence of recombinant CYP2C19 (Cypex/XenoTech). NADPH, a required cofactor for CYP450 metabolism, was provided by the NADPH Regenerating System, Solutions A (BD Biosciences) and B (BD Biosciences). Compound stock solution was initially prepared in 100% DMSO and subsequently diluted in acetonitrile for the assay. The pH of the reactions was maintained at ˜7.4 with potassium phosphate buffer (BD Biosciences). The reaction was started after adding NADPH to the reaction plate containing the enzyme, the luciferin substrate, and compound. The reaction was incubated for 30 minutes at 37° C. with shaking. Luciferin Detection Reagent was added after the incubation to stop the reaction and initiate a stable glow-type luminescent signal. The luminescence of the metabolite formed, Luciferin, was recorded with the Infinite M200 (Tecan US). All reactions were run in duplicate, except negative controls (no enzyme) which were performed as single reactions. The background luminescence obtained from the negative control reactions was substracted from the luminescence values obtained for the positive reaction duplicates. The percent of inhibition was calculated by the formula: 100−[RLU at 10 μM/RLU at 0 μM*100].

CYP450 Inhibition Profiling—Inhibition of Drug-Metabolizing CYP2D6—

Evaluation of the compound as inhibitor of the activity of CYP2D6 was assessed using the P450-Glo™ Assay kit (Promega) in the presence of recombinant CYP2D6 (Cypex/XenoTech). NADPH, a required cofactor for CYP450 metabolism, was provided by the NADPH Regenerating System, Solutions A (BD Biosciences) and B (BD Biosciences). Compound stock solution was initially prepared in 100% DMSO and subsequently diluted in acetonitrile for the assay. The pH of the reactions was maintained at ˜7.4 with potassium phosphate buffer (BD Biosciences). The reaction was started after adding NADPH to the reaction plate containing the enzyme, the luciferin substrate, and compound. The reaction was incubated for 30 minutes at 37° C. with shaking. Luciferin Detection Reagent was added after the incubation to stop the reaction and initiate a stable glow-type luminescent signal. The luminescence of the metabolite formed, Luciferin, was recorded with the Infinite M200 (Tecan US). All reactions were run in duplicate, except negative controls (no enzyme) which were performed as single reactions. The background luminescence obtained from the negative control reactions was substracted from the luminescence values obtained for the positive reaction duplicates. The percent of inhibition was calculated by the formula: 100−[RLU at 10 μM/RLU at 0 μM*100].

Representative Biological Activity of Exemplary Compounds of the Present Technology

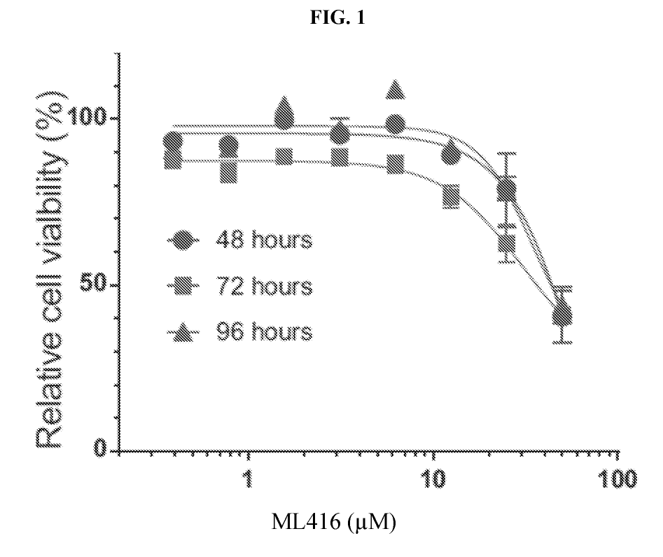

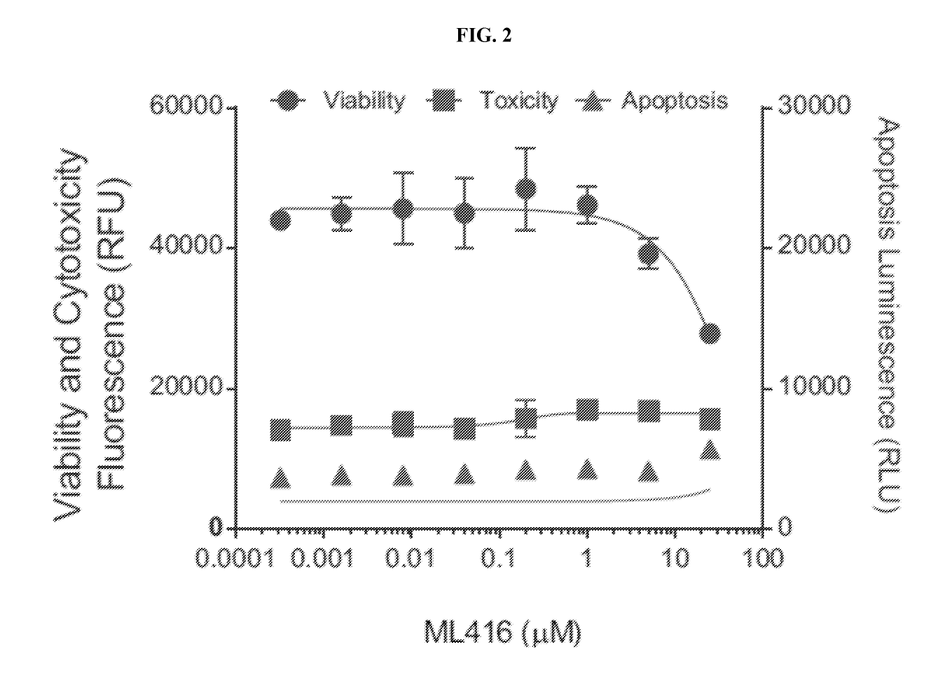

ML416 was initially evaluated for cytotoxicity, as a cytotoxic compound may misleadingly exhibit broad-spectrum antiviral activity. Vero76 cells were plated in 96-well plates and incubated 48, 72, and 96 hours in the presence of ML416 at various concentrations. ML416 did not show apparent cytotoxicity up to 12.5 μM (FIG. 1). The CC₅₀ values were 74.1, 31.0 and 34.6 μM after 2, 3, and 4 days exposure respectively, resulting the Selective Index 50 (SI₅₀) greater than 100 (CC₅₀ at Day3/EC₅₀ of CHIKV=106.9). This indicates that ML416 is not toxic to cells at the effective concentrations. ML416 was further tested in viability, cytotoxicity and/or apoptosis assays with HEK 293T cells and no significant cytotoxicity and apoptosis was induced in the presence of ML416 up to 25 μM for the four days exposure (FIG. 2).

ML416 was profiled against a panel of viruses, the results of which are shown below in Table 2. For titration reduction assays, the progeny virus titers were compared to the controls (DMSO treated). For dose-response assays, cells were pretreated for two hours prior to infection and the luciferase activity was measured 16 hours later. Blank spaces indicate that while final data is not yet ready, initial results have shown activity with the indicated viral strain.

TABLE 2

| | | Log titer | EC₅₀ |
|---|---|---|---|
| Virus family | Virus | inhibition at 5 μM** | (μM) |
| Rhabdovidae | pVSV-luc | | 0.1950* |
| Togaviridae | VEEV TC-83 | −2.536 ± 0.018 | 0.35 |
| | VEEV TrD | −4.768 | 0.46 |
| | VEEV V3526-luc | | 0.17 |
| | CHIKV S17 | −4.024 | 0.29 |
| | WEEV | | 0.96 |
| Paramyxoviridae | RSV Long | −2.27 ± 0.1 | |
| Orthomyxoviridae | Influenza virus A | −3.03 | |
| | (H1N1) | | |
| Coronaviridae | SARS-CoV | | |
| Arenaviridae | LCMV-ARM | −3.466 | |
| Retroviridae | HIV-1 | | 94% |
| | | | inhibition |
| | | | at 10 μM |
| Herpesviridae | HSV-2 | 0.01 ± 0.43 | |
| Picornaviridae | EV-71, MP4 | 11.1 | NT |
| | EMCV MM | >25 | NT |
| Flaviviridae | JEV | −1.3 ± −0.43 | 1.9 |
| | YFV 17D | −2.32 | |
| | WNV/NY99 | −2.05 ± −0.09 | 2.4 |
| Filoviridae | Ebola virus-GFP | | 0.26 |

*Luciferase-tagged virus assay
**Negative values mean a decrease in progeny virus titer compared to the mock treated controls.
NT = Not tested
pVSV-luc = Vesicular stomatitis virus
RSV = Respiratory syncytial virus
JEV = Japanese Encephalitis virus
HSV-2 = Herpes simplex virus type-2
YFV 17D = Yellow Fever virus 17D
WNV/NY99 = West Nile virus/strain
EV-71 = human enterovirus 71
EMCV = Encephalomyocarditis virus
WEEV = West equine encephalitis virus California
SARS-CoV = Severe acute respiratory syndrome coronavirus
LCMV-ARM = Lymphocytic Choriomeningitis Virus ARM strain

As shown in Table 2, ML416 showed an antiviral effect against a broad-spectrum of viruses with different sensitivity to each virus. Alphaviruses, LCMV, and IFNV were most sensitive to ML416. The treatment of ML416 at 5 μM resulted in a greater than 4 Log reduction in the progeny virus titers of alphaviruses tested, and greater than 3 Log reduction for influenza virus (IFNV) and LCMV. RSV, WNV, YFV 17D were sensitive as well with—2 Log titer reduction; however, JEV was less sensitive that the others tested (˜1.3 Log reduction). HIV-1 infection was sensitive as well, with 94% reduction in viral infectivity using TZM-bl reporter system measuring luciferase activity with 10 mM of ML416.

EC₅₀ measurement was used to assess ML416's antiviral activity using a dose-response format. The replication of pVSV-luc or EBO-GFP was sensitive to the treatment of ML416 with EC₅₀ values of 0.19 mM and 0.30 mM, respectively. From these experiments, it was found that ML416 has a broad-spectrum antiviral effect with different sensitivities depending on virus. This result implies that the antiviral mechanism of ML416 has specificity to certain type of viruses.

ML416 was also tested against two strains of Zika virus, where the EC₅₀ and cytotoxicity results are provided in Table 3. As shown by Table 3, ML416 possesses an antiviral effect for Zika virus.

TABLE 3

| | | | Cell | | EC₅₀ | CC₅₀ μM |
|---|---|---|---|---|---|---|
| Compound | Structure | Virus | Type | MOI | (μM) | (Vero76) |
| ML416 | 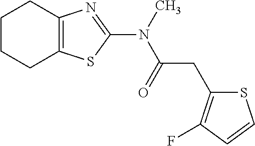 [SMILES: CN(C(=O)Cc1sccc1F)c1nc2c(s1)CCCC2] | JEV WNV/NY99 ZIKV/PRVABC59 ZIKV/MR766 | Vero76 Vero76 Vero76 Vero76 | 0.05 0.01 0.05 0.05 | 1.9 2.4 2.2 1.1 | >50 >50 >50 >50 |

WNV = west nile virus/strain
JEV = Japanese encephalitis virus
ZIKV = Zika Virus/strain

To test whether ML416 could interfere with cellular metabolism for its antiviral activity, the difference in cellular metabolites was measured using a metabolomics approach. HEK 293T cells were treated either with 5 mM of ML416 or with DMSO for 18 hours and the cellular metabolites were analyzed and compared using GC-MASS spectrophotometry. The treatment of ML416 changed the abundance of cellular metabolites significantly (Table 4). For example, the amounts of L-glutamic acid and fumaric acid differed by 0.09- and 11-fold when ML416-treated group was compared to mock-treated cells. Some metabolites showed changes that are more evident; exclusively detected only in one group (Table 5). For example, dihydroorotic acid (DHO) and orotic acid were found only in ML416-treated groups. DHO and orotic acid are metabolites that are related with de novo pyrimidine synthesis, suggesting that ML416 inhibits pyrimidine synthesis after the synthesis of orotic acid from DHO. Consistent with this result, free uridine was not detected in the ML416-treated groups.

TABLE 4
Metabolites with significant abundance changes between sample groups.

| Name | CAS | Fold Change ** | p-value |
|---|---|---|---|
| Pyruvic acid | 127-17-3 | 1.60 | 1.29E−04 |
| D-(−)-Lactic acid | 10326-41-7 | 0.76 | 2.02E−04 |
| L-Aspartic acid | 56-84-8 | 2.07 | 2.02E−02 |
| Oxalic acid | 144-62-7 | 0.66 | 1.08E−02 |
| L-Proline | 147-85-3 | 0.28 | 1.25E−03 |
| L-Isoleucine | 73-32-5 | 0.45 | 2.54E−03 |
| Glycine | 56-40-6 | 2.80 | 0.00 |
| Fumaric Acid | 110-17-8 | 11.00 | 0.00 |
| L-Threonine | 6028-28-0 | 0.54 | 3.01E−02 |
| DL-Phenylalanine | 150-30-1 | 0.71 | 4.55E−02 |
| L-Asparagine | 70-47-3 | 0.41 | 6.74E−05 |
| L-Glutamic acid | 56-86-0 | 0.09 | 0.00E+00 |
| Phenylalanine | 150-30-1 | 0.40 | 3.16E−04 |
| Lauric acid; Dodecanoic acid | 143-07-7 | 0.73 | 1.45E−03 |
| DL-Ornithine | 616-07-9 | 0.53 | 2.70E−06 |
| Citirc acid | 77-92-9 | 0.62 | 1.67E−04 |
| L-Tyrosine | 60-18-4 | 0.86 | 2.21E−02 |
| L-Lysine | 56-87-1 | 2.76 | 9.44E−05 |
| Tryptophan | 73-22-3 | 1.23 | 1.56E−04 |
| 2-(Dimethylamino)ethyl neopentyl | NA | 1.49 | 6.98E−03 |
| carbonate* | | | |
| 3-((carboxymethyl)(methyl)amino)- | NA | 0.46 | 0.00 |
| 3-oxopropanoic acid | | | |
| Dihydromuconic acid; trans-3- | 4436-74-2 | 0.23 | 0.00 |
| Hexenedioic acid* | | | |
| 3,3-diethoxy-2-hydroxypropyl | NA | 0.58 | 1.89E−05 |
| dihydrogen phosphate* | | | |
| Ribitol* | 488-81-3 | 0.66 | 1.97E−04 |
| O-phosphorylethanolamine; 2- | 1071-23-4 | 1.35 | 2.02E−02 |
| aminoethyl dihydrogen phosphate* | | | |
| meso-Erythritol; Erythritol* | 149-32-6 | 0.69 | 1.80E−03 |
| 1-phenylpropan-2-amine; | 300-62-9 | 0.80 | 2.85E−03 |
| Amphetamine; Fenopromin* | | | |
| 3-Glycerophosphate* | 57-03-4 | 1.71 | 4.08E−03 |
| Scyllo-Inositol* | 488-59-5 | 1.23 | 0.00 |
| Adenine* | 73-24-5 | 1.37 | 3.23E−02 |
| Niacinamine* | 98-92-0 | 0.74 | 1.11E−02 |
| L-Norvaline* | 6600-40-4 | 0.52 | 3.16E−02 |
| 3-hydroxy-2-methylpropanoic acid; 3- | 2068-83-9 | 0.70 | 5.18E−03 |
| Hydroxyisobutyric acid* | | | |
| Ethylcholine mustard* | 4669-20-9 | 0.49 | 4.90E−02 |

Note:
Each of these metabolites was detected in more than 75% of samples in each sample group by GC × QC-TOF MS.
*These compounds have not been confirmed by metabolite standards.
** Fold change was defined as the ratio of average peak abundance of a metabolite in sample group ML416-treated divided by the average peak abundance of the same metabolite in the DMSO-treated sample group.

TABLE 5
Difference in cellular metabolites by the treatment of ML416

| Groups | Name | CAS | p-value |
|---|---|---|---|
| Metabolites present in | dl-Dihydroorotic acid | 6202-10-4 | 4.11E−04 |
| ML416-treated cells, | Orotic acid | 6784-70-9 | 2.26E−03 |
| absent in mock-treated | l-Serine | 56-45-1 | 1.52E−02 |
| cells | Citrulline | 627-77-0 | 4.11E−05 |
| | Alloxanoic acid* | 470-44-0 | 4.11E−05 |
| | 2-Ketoisovaleric acid* | 759-05-7 | 4.11E−05 |
| Metabolites absent in | Uridine | 58-96-8 | 4.11E−05 |
| ML416-treated cells, | D-(−)-Erythrofuranose,(isomer)* | NF | 4.16E−04 |
| present in mock-treated | (3R,4R)-tetrahydrofuran-2,3,4- | 95-44-3 | 4.11E−05 |
| cells | triol* | | |
| | Dihydromuconic acid; trans-3- | 4436-74-2 | 1.52E−02 |
| | Hexenedioic acid* | | |
| | Uridine phosphate* | 58-97-9 | 4.11E−04 |
| | Methanesulfmic acid* | 17696-73-0 | 4.11E−04 |
| | N-Acetyl-L-glutamic acid* | 1188-37-0 | 4.11E−05 |
| | Putrescine; 1,4-Diaminobutane* | 110-60-1 | 4.11E−05 |
| | Pidolic acid* | 149-87-1 | 3.30E−03 |
| | 2-Hydroxyglutaric acid | 2889-31-8 | 2.32E−03 |

*The compounds that have not been confirmed by using standard compounds.

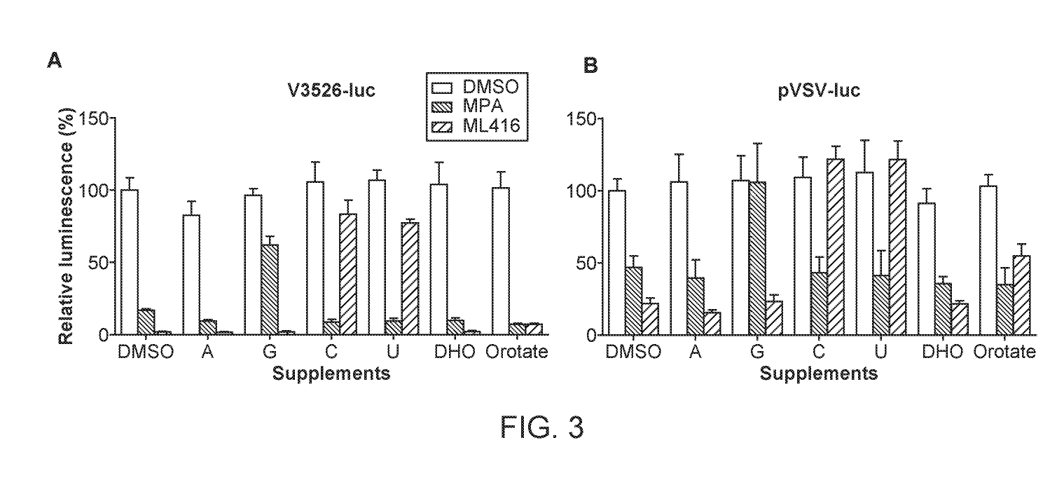

To validate this finding, a series of experiments was performed to see if the antiviral effect of ML416 could be neutralized by the addition of exogenous pyrimidines. he virus replication was measured in HEK 293T cells treated with mycophenolic acid (MPA) or ML416 in the presence of various exogenous nucleosides, and then compared with the virus replication in the controls (FIG. 3). In MPA-treated groups, the antiviral effect of MPA was completely reversed when guanosine was supplemented in the culture: from 47% to 105% and 17% to 62% for pVSV-luc and V3526-luc, respectively. This result was consistent with MPA inhibition of inosine-5′-monophosphate dehydrogenase (IMPDH) as its antiviral mechanism, which is a key enzyme to synthesize guanosine de novo. Similarly, the antiviral effect of ML416 greatly decreased when the cells were supplemented with pyrimidines, cytidine or uridine. For example, while 5 mM of ML416 decreased the replication of V3526-luc to 1.9% compared to the control, the addition of cytidine or uridine restored the viral replication 83.4 or 77.5% compared to the control, respectively.

Two pyrimidine synthesis intermediates, DHO and orotic acid, were also tested to confirm the metabolomics results with the accumulation of the molecules in the ML416 treated group. The addition of DHO did not affect the antiviral activity of ML416 at all, suggesting ML416 inhibits a downstream step from DHO. Orotic acid showed a moderate reversion effect to ML416 (21.9% to 55.0% and 1.9% to 7.1% for pVSV-luc and V3526-luc, respectively).

Induction of Innate Immune Genes by ML416 without Virus Infection or Type 1 Interferons.

It has been reported that the inhibition of pyrimidine biosynthesis could amplify the cellular response to ssRNA via type 1 IFN system; however, we questioned this as Vero 76, the primary cell line for the antiviral activity testing for ML416 and its derivatives, is known to be deficient in type 1 IFN production.

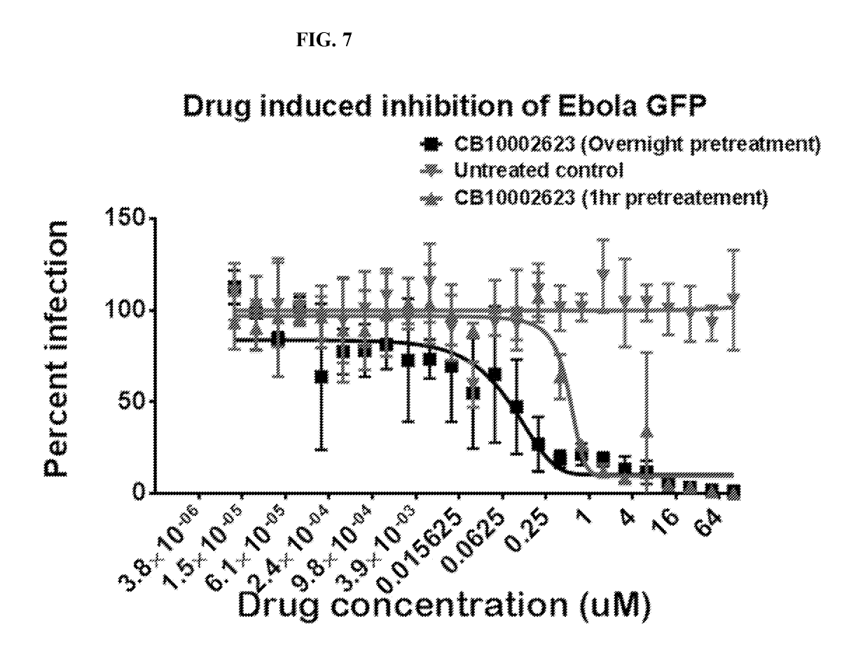

To test whether ML416 induced cellular immune response without type 1 IFN, the changes in host gene expression was examined after the treatment of a compound of the present technology (CB10002623, illustrated below; see also Table 8, entry 6 below and FIG. 7), without addition of ssRNA.

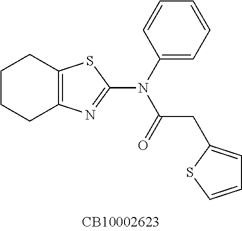 [SMILES: O=C(Cc1cccs1)N(c1ccccc1)c1nc2c(s1)CCCC2]

Human HEp-2 cells were treated with 5 μM of CID:70698683 or DMSO for 18 hours and then the cellular mRNAs were subjected to a DNA microarray assay. Ninety-two genes were up-regulated and one hundred forty five genes were down-regulated by greater than two-fold difference (GEO accession ID: GSE72167). Among these changes, certain sets of genes that are involved in the interferon pathways were clearly upregulated, including RIG-I (4.63-fold) and OASL (3.76-fold increase). Some interferons-stimulated genes (ISGs), e.g., GBP2, ISG20, IFI44, IRF9 or IFIT1, were also upregulated significantly (3.5, 2.85, 2.83, 2.4 or 2.3, respectively). While the ISGs were upregulated, the expressions of interferons were not upregulated (0.9˜1.1 fold changes); rather, the expression of IFN-ε decreased in half. These findings are consistent with the induction of ISGs without functional IFNs.

This finding was further validated with a real-time PCR and ELISA assay. First, we sought to understand whether ML416 simply amplified cellular innate immune responses after virus infection or if ML416 was able to establish an antiviral state without an external interferon inducer, such as virus infection. To test this, the induction of innate immune response genes by ML416 was measured in mock or pVSV-luc infected HEK 293T cells.

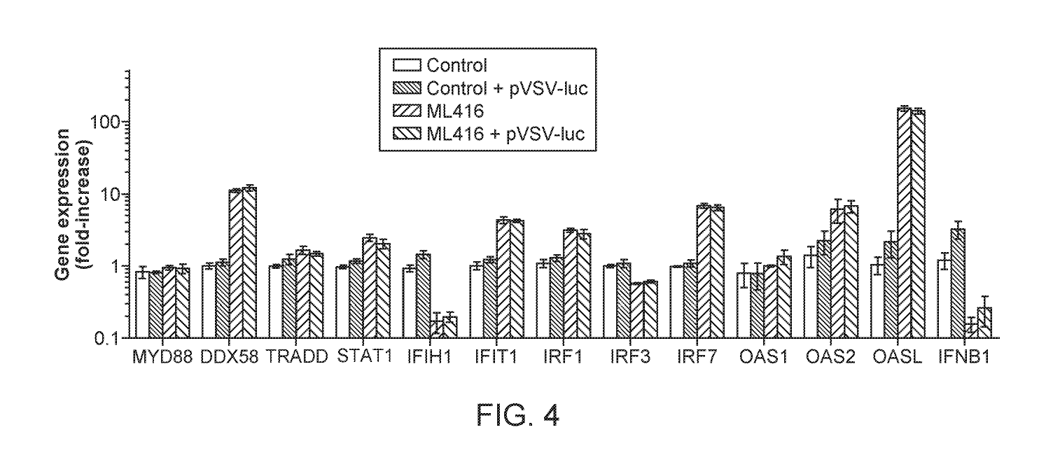

As shown in FIG. 4, the induction of innate immune genes by ML416 was independent of virus infection. First, it confirmed that the treatment of ML416 induces only certain genes in the interferon pathway. For example, the expressions of MYD88 and OAS1 did not show any changes by ML416; however, RIG-I, IFIT1, IRF7, and OAS2 genes were upregulated more than 10-fold by ML416. More interestingly, the induction of the genes by ML416 was independent of virus infection. The treatment of ML416 in mock- or virus-infected cells (ML416 vs. ML416+pVSV-luc) resulted in a same level of gene expression for the tested genes. The induction of the ISGs by ML416 was much stronger than the induction by pVSV-luc infection. pVSV-luc increased the expression of innate immune genes, such as IFIH1, IFIT1, OAS2, OASL and IFNB1 by about 2˜4 fold changes, indicating that HEK 293T cells are responsive to the infection of the virus. However the effect was much weaker compared to that of ML416. Interestingly, ML416 repressed the expression of IFIH1 and IFNB1. These repressions were not seen in virus-infected groups (“Control+pVSV-luc”), where the expression of the genes increased.

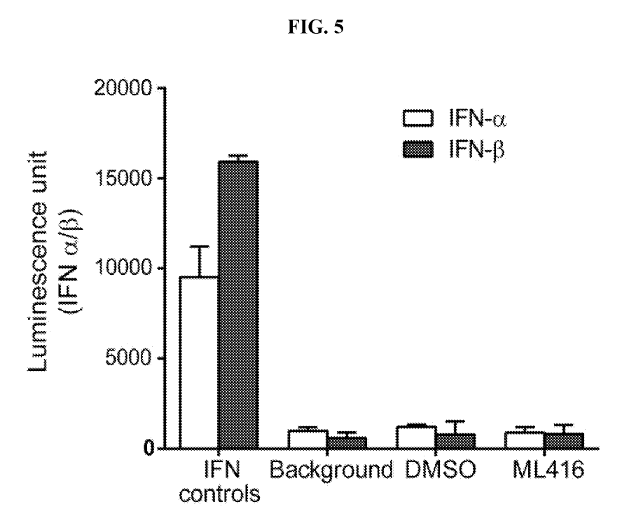

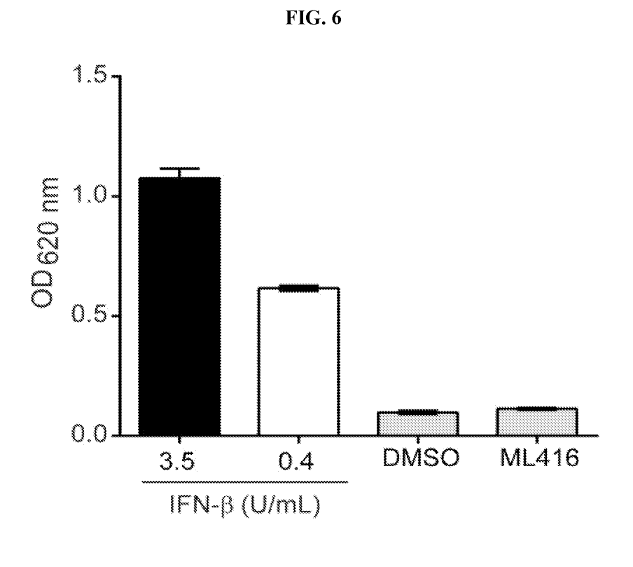

The lack of induction of type 1 IFNs by ML416 was confirmed at the cytokine level with ELISA and a cell-based reporter assay. HEK 293T cells were treated with 5 mM of ML416 for 18 hours then the IFN α/β in the cell culture supernatant was detected in an IFN ELISA assay (FIG. 5) or in a reporter assay (HEK-Blue™ IFN-α/β cells). HEK-Blue™ IFN-α/β cells directly respond to IFN α/β and express the secreted alkaline phosphatase (SEAP) via the activation of IRF9. In both assays, no measurable quantity of IFNs was detected compared to the controls, indicating the lack of IFN α/β induction by the treatment of ML416. HEK-Blue™ IFN-α/β cells were treated directly with ML416 to test whether ML416 can activate the IRF9 pathway or produce IFN as in an autotroph mode (FIG. 6). The treatment of ML416 did not induce SEAP activity compared to the control as well, indicating no direct activation through IFNAR or any other receptor that can use IRF9.

In summary, these data indicate that ML416 induces expression of certain sets of innate immunity genes without the induction of type 1 IFNs.

Cell Line Specificity of ML416.

Since ML416 inhibits the pyrimidine synthesis and induces the antiviral state of the host cells, we questioned whether the antiviral effect and mechanism of ML416 is cell type-dependent. To address this question, the antiviral activity of ML416 in several cell lines was measured by determining an EC₅₀ value for each. The cell lines include HEK 293T (human embryonic kidney), SY-SH5S (human bone marrow derived neuroblast), Vero76 (African green monkey kidney fibroblast), BHK C21 (hamster kidney fibroblast), Neuro2A (mouse neuroblast), and NIH3T3 (mouse fibroblast).

The antiviral activities of ML416 in various cell lines are summarized in Table 6 as a function of EC₅₀ value. As a control for the experiment, monensin was used, which is known to inhibit virus replication by hampering the acidification of endosome during virus entry. The antiviral activities of monensin were very close to each other in all cell lines tested in this experiment. The EC₅₀ values were within a range between 0.02 and 0.25 for V3526-luc, and between 0.17 and 0.56 mM for pVSV-luc. This result shows that endocytosis is a critical pathway for the viruses in the cell lines and monensin worked equally in the cell lines.

In contrast to monensin, the antiviral activities of ML416 were cell line or species-dependent. The EC₅₀ values of ML416 in HEK 293T or Vero76 were between 0.15 mM and 0.68 mM for both V3526-luc and pVSV-luc, implying a strong antiviral activity against both viruses in these cell lines. Contrary to this, no antiviral activities of ML416 were detected in the mouse cell lines (Neuro 2A and NIH 3T3) we tested (EC₅₀>50 for both viruses). Interestingly, ML416 still showed a moderate effect in BHK cells, a hamster cell line. These data clearly indicate that ML416 is cell line-dependent, with ML416 showing the strongest antiviral effect in human cells and much less effective in mouse cells.

TABLE 6
Antiviral activity of ML416 in various cell lines

| | EC₅₀ (μM) of | | | |
|---|---|---|---|---|
| | Monensin | | ML416 | |
| | V3526-luc | pVSV-Gp | V3526-luc | pVSV-Gp |
| HEK 293T | 0.08 | 0.19 | 0.15 | 0.17 |
| | 0.03 | 0.74 | | |
| SY-SH5S | 0.02 | 0.17 | 0.49 | 3.82 |
| | | 0.48 | | |
| Vero76 | 3.87 | 0.56 | 0.17 | 0.20 |
| | | 2.57 | 0.25 | 0.68 |
| BHK | 0.09 | 0.50 | 1.12 | 8.26 |
| | 0.24 | 0.19 | 0.92 | 7.16 |
| Neuro2A | 0.25 | 0.22 | >50 | >50 |
| | 0.19 | 0.23 | | |
| | | 0.29 | | |
| NIH 3T3 | 0.02 | 0.24 | >50 | >50 |
| | | 0.67 | | |

Note:
In Table 6, each number represents EC₅₀ evaluated from a dose-response study with concentrations starting from 25 μM by a five-fold dilution, triplicates for each, in a 96-well format. For assays with HEK 293T, Neuro 2A, and SH-SY5Y cells, 24,000 cells and 2,400 TCID₅₀ units of virus per well was used. For Vero 76 and BHK cells, 12,000 cells and 1200 TCID₅₀ units of virus per well was used. For NIH3T3, 24,000 cells and 20,000 TCID₅₀ units of virus per well was used. EC₅₀ values were calculated using XLfit (IDBS) formula 205, a 4-parameter Levenburg-Marquardt algorithm with maximum and minimum limits set at 100 and 0, respectively.

Various other compounds of the present technology were examined for their biological activity, as shown in the Tables below.

TABLE 7
Benzothiazole modifications

| | 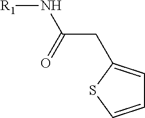 | CHIKV (S27 strain) CPE Assay Potency mean (μM) | VERO Cell Cyto- toxicity Assay mean (μM) | VEEV (TC83 strain) CPE Assay Potency mean (μM) |
|---|---|---|---|---|
| Entry | R₁ | EC₅₀ | CC₅₀ | EC₅₀ |
| 1 | 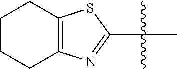 [SMILES: *c1nc2c(s1)CCCC2] | 4.9 | >50.0 | 5.1 |
| 2 | 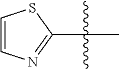 [SMILES: *c1nccs1] | 28.2 | >50.0 | >50.0 |
| 3 | 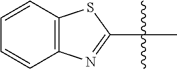 [SMILES: *c1nc2ccccc2s1] | 39.7 | >50.0 | >50.0 |

TABLE 8
Structure-activity relationships associated with substitution on the NH group

| | 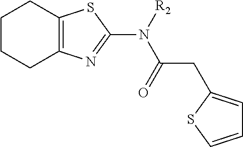 | CHIKV (S27 strain) CPE Assay Potency mean (μM) | VERO Cell Cytotoxicity Assay mean (μM) | VEEV (TC83 strain) CPE Assay Potency mean (μM) |
|---|---|---|---|---|
| Entry | R₂ | EC₅₀ | CC₅₀ | EC₅₀ |
| 1 | H | 4.9 | >50.0 | 5.1 |
| 2 | CH₃ | 4.5 | >50.0 | 1.9 |
| 3 | CH₂CH₃ | NT | >50.0 | 1.1 |
| 4 | n-propyl | 3.0 | >50.0 | 1.0 |
| 5 | isopropyl | >50.0 | >50.0 | 27.5 |
| 6 | phenyl | 0.7 | >50.0 | 0.5 |
| 7 | benzyl | 7.5 | >50.0 | 2.8 |

NT = not fully tested

TABLE 9
Substitution in the amide moiety

| | 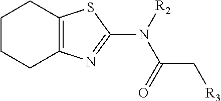 | | CHIKV (S27 strain) CPE Assay Potency mean (μM) | VERO Cell Cytotoxicity Assay mean (μM) | VEEV (TC83 strain) CPE Assay Potency mean (μM) |
|---|---|---|---|---|---|
| Entry | R₂ | R₃ | EC₅₀ | CC₅₀ | EC₅₀ |
| 1 | H | 2- | 4.9 | >50.0 | 5.1 |
| | | thiophene | | | |
| 2 | CH₃ | phenyl | 3.1 | >50.0 | 2.5 |
| 3 | CH₃ | 2-CF₃- | >50.0 | >50.0 | 0.9 |
| | | phenyl | | | |
| 4 | CH₃ | 3-CF₃- | >50.0 | >50.0 | 0.9 |
| | | phenyl | | | |
| 5 | CH₃ | 4-CF₃- | >50.0 | >50.0 | >50.0 |
| | | phenyl | | | |
| 6 | CH₃ | 2-OCH₃- | 1.0 | >50.0 | 0.9 |
| | | phenyl | | | |
| 7 | CH₃ | 2-OEt- | 0.7 | >50.0 | 0.4 |
| | | phenyl | | | |
| 8 | CH₃ | 4-OEt- | >50.0 | >50.0 | >50.0 |
| | | phenyl | | | |
| 9 | CH₃ | 2- | 9.3 | >50.0 | 9.1 |
| | | CH₂OCH₃- | | | |
| | | phenyl | | | |
| 10 | CH₃ | 2-CH₃- | 3.6 | >50.0 | 1.0 |
| | | phenyl | | | |
| 11 | CH₃ | 3-CH₃- | 2.8 | >50.0 | 1.3 |
| | | phenyl | | | |
| 12 | CH₃ | 4-CH₃- | 4.1 | >50.0 | 0.9 |
| | | phenyl | | | |
| 13 | CH₃ | 2,5- | 8.9 | >50.0 | 11.7 |
| | | (CH₃)₂- | | | |
| | | phenyl | | | |
| 14 | i-propyl | 2-OEt- | >50.0 | >50.0 | 15.9 |
| | | phenyl | | | |
| 15 | 4-pyran | 2-OEt- | >50.0 | >50.0 | 12.5 |
| | | phenyl | | | |
| 16 | (CH₂)₂OCH₃ | 2-OEt- | 10.9 | >50.0 | 10.9 |
| | | phenyl | | | |
| 17 | phenyl | 2-OCH₃- | 0.5 | >50.0 | 0.7 |
| | | phenyl | | | |
| 18 | CH₃ | —(CH₂)-2- | >50.0 | | |
| | | thiophene | | | |

TABLE 10

| | 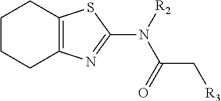 | | CHIKV (S27 strain) CPE Assay Potency mean (μM) | VERO Cell Cytotoxicity Assay mean (μM) | VEEV (TC83 strain) CPE Assay Potency mean (μM) |
|---|---|---|---|---|---|
| Entry | R₂ | R₃ | EC₅₀ | CC₅₀ | EC₅₀ |
| 18 | phenyl | 2-OEt- | 14.7* | >50.0 | 0.4 |
| | | phenyl | | | |
| 19 | phenyl | 2-CF₃- | 1.2 | >50.0 | 1.2 |
| | | phenyl | | | |
| 20 | 2-OCH₃- | 2-OEt- | >50.0 | >50.0 | >50.0 |
| | phenyl | phenyl | | | |
| 21 | 3-OCH₃- | 2-OEt- | 4.1 | >50.0 | 1.7 |
| | phenyl | phenyl | | | |
| 22 | 4-OCH₃- | 2-OEt- | >50.0 | >50.0 | 4.6 |
| | phenyl | phenyl | | | |
| 23 | 2-thiazole | 2-OEt- | >50.0 | >50.0 | >50.0 |
| | | phenyl | | | |
| 24 | 3-pyridyl | 2-OEt- | 10.6 | | 2.5 |
| | | phenyl | | | |
| 25 | phenyl | 2-pyridyl | >50.0 | 33.2 | >50.0 |
| 26 | phenyl | 2-furyl | 8.2 | 25.8 | 6.8 |
| 27 | phenyl | 5′-Br-2- | 0.05 | 17.2 | 0.02 |
| | | thiophene | | | |
| 28 | phenyl | 3′-Br-2- | 0.05 | 22.4 | 0.02 |
| | | thiophene | | | |
| 29 | phenyl | 5′-CH₃-2- | 0.11 | 7.8 | 0.03 |
| | | thiophene | | | |
| 30 | CH₃ | 4′-CH₃-2- | 0.09 | 36.4 | 0.02 |
| | | thiophene | | | |
| 31 | CH₃ | 4′-OCH₃-2 | 0.14 | 36.1 | 0.05 |
| | | thiophene | | | |
| 32 | CH₃ | 4′-F-2- | 10.2 | 22.5 | 5.1 |
| | | thiophene | | | |
| 33 | CH₃ | 3′-F-2- | 0.3 | 41.4 | 0.4 |
| | | thiophene | | | |
| 34 | CH₃ | 3′-CF₃-2- | >50.0 | 6.4 | NT |
| | | thiophene | | | |
| 35 | CH₃ | 5′-CF₃-2- | >50.0 | >50.0 | >50.0 |
| | | thiophene | | | |

*indicates result is subject to futher testing and may be a lower value

TABLE 11

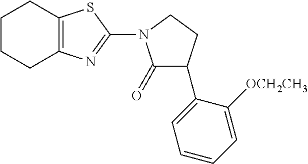 [SMILES: CCOc1ccccc1C1CCN(c2nc3c(s2)CCCC3)C1=O]

CHIKV EC₅₀ > 50 mM
VEEV EC₅₀ = 4.5 mM
CC₅₀ > 50 mM

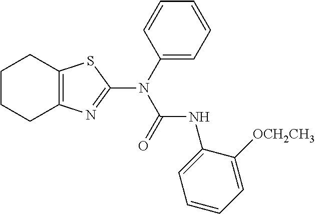 [SMILES: CCOc1ccccc1NC(=O)N(c1ccccc1)c1nc2c(s1)CCCC2]

CHIKV EC₅₀ = 12.2 mM
VEEV EC₅₀ = 10.0 mM
CC₅₀ > 50 mM

A titer reduction assay for several compounds in the series was performed, where the resulting data is provided in Table 12. The assay was carried out at a 5 μM concentration of each compound. There are no units for the values—they are reported as a log value. For example, for A in Table 12, CHIKV, is reported as a log 0.61. A larger number generally indicates better performance than the control (DMSO). For reference, a compound with a value of 2 is 100-fold better than control. A value of 3 is 1000-fold better and so on.

TABLE 12

| Log titer | | | | | | | | |
|---|---|---|---|---|---|---|---|---|
| reduction | | CID | | | | | | |
| at 5 μM | DMSO | A | B | C | D | E | F | G |
| CHIKV S27 | 0 | 0.612 | 4.024 | 3.62 | 2.182 | 3.161 | 2.495 | 5.179 |
| LCMV | 0 | 0.085 | 3.466 | 3.014 | NT | NT | NT | NT |
| ARM | | | | | | | | |
| Trinidad | 0 | 1.765 | 4.768 | 3.741 | 2.716 | 3.411 | 3.198 | 4.754 |
| Donkey | | | | | | | | |
| YFV 17D | 0 | NT | 2.32 | 2.588 | NT | NT | NT | NT |
| A/NY/ | 0 | 4.949 | 4.949 | 3.075 | NT | NT | NT | NT |
| 18/2009 | | | | | | | | |

NT in the table = not tested

### Compounds of Table 7:

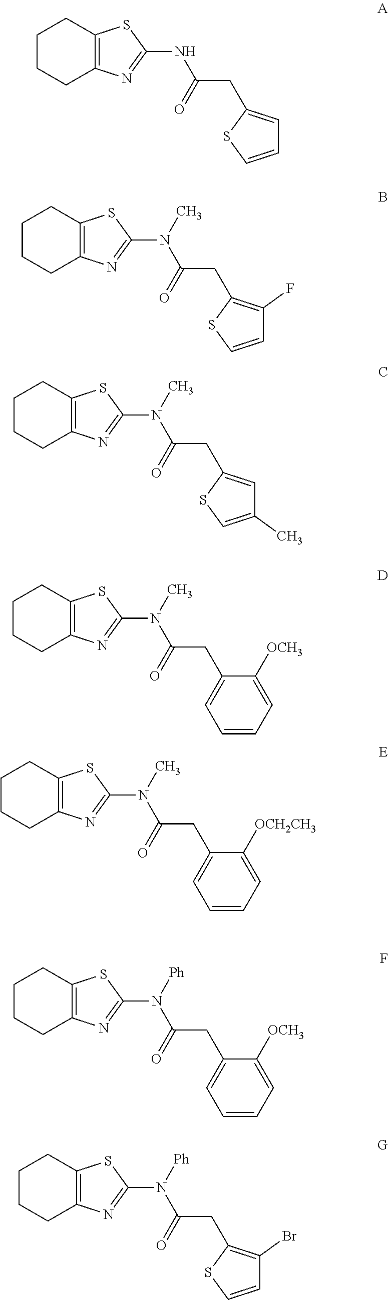

Virus names in Table 7:

CHIKV S27=chikungunya virus, strain S27

LCMV ARM=Lymphocytic Choriomeningitis Virus

Trinidad donkey=VEEV, strain trinidad donkey

YFV 17D=Yellow Fever Virus, strain 17D

A/NY/18/2009=Influenza A virus

In addition, FIG. 7 provides the inhibition of Ebola by the compound of the present technology shown below, identified in FIG. 7 as CB10002623.

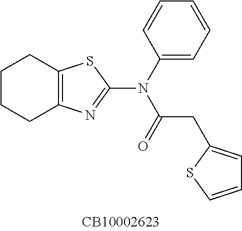 [SMILES: O=C(Cc1cccs1)N(c1ccccc1)c1nc2c(s1)CCCC2]

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology includes, but is not limited to, the following lettered paragraphs:
A. A compound according to Formula I

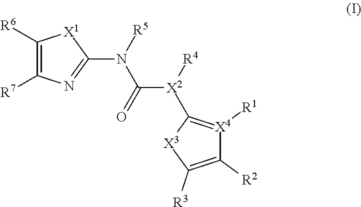

or a solvate and/or pharmaceutically acceptable salt thereof; wherein
X¹ is NH, S, or O;
X² is N, CH, or C-(unsubstituted alkyl);
X³ is S, O, or —C(R⁸)═C(R⁹)—;
X⁴ is N or C;
R¹, R², R³, R⁸ and R⁹ are each independently H, halo, cyano, trifluoromethyl, nitro, pentafluorosulfanyl, or a substituted or unsubstituted alkyl, alkoxy, aryl, aryloxy, alkynyl, cycloalkyl, heterocyclylalkyl, alkanoyl, alkanoyloxy, aryloyl, aryloyloxy, carboxylate, or ester group; provided that when X⁴ is N, then R¹ is absent;
R⁴ is H or unsubstituted alkyl;
R⁵ is H, or a substituted or unsubstituted alkyl, alkoxy, aryl, heteroaryl, alkynyl, cycloalkyl, heterocyclyl, heterocyclylalkyl, alkanoyl, or aryloyl group; or R⁴ and R⁵ taken together are a substituted or unsubstituted C₂-C₄ alkylenyl group;
R⁶ and R⁷ are each independently H or unsubstituted alkyl, or R⁶ and R⁷ taken together form a substituted or unsubstituted aryl, or R⁶ and R⁷ taken together are a substituted or unsubstituted C₃-C₅ alkylenyl or —(CH₂)ₓ—O—(CH₂)_{y}— group, where x and y are each independently 1, 2, or 3 provided that when x is 3, y is 1, and when y is 3, x is 1.
B. The compound of Paragraph A, wherein X² is N or CH.
C. The compound of Paragraph A or Paragraph B, wherein R⁴ is H, or R⁴ and R⁵ taken together are a substituted or unsubstituted C₂-C₄ alkylenyl group.
D. The compound of any one of Paragraphs A-C, wherein R⁶ and R⁷ taken together form a substituted or unsubstituted aryl or R⁶ and R⁷ taken together are a substituted or unsubstituted C₃-C₅ alkylenyl.
E. The compound of any one of Paragraphs A-D, where the compound is according to Formula II

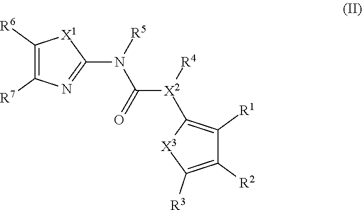

wherein
X² is N or CH;
R⁴ is H; or R⁴ and R⁵ taken together are a substituted or unsubstituted C₂-C₄ alkylenyl group;
R⁶ and R⁷ taken together form a substituted or unsubstituted aryl or R⁶ and R⁷ taken together are a substituted or unsubstituted C₃-C₅ alkylenyl.
F. The compound of any one of Paragraphs A-E, wherein R⁵ is a substituted or unsubstituted alkyl, a substituted or unsubstituted phenyl, or an unsubstituted heterocyclyl group.
G. The compound of any one of Paragraphs A-F, wherein R⁵ is a substituted or unsubstituted alkyl, a substituted or unsubstituted phenyl, or an unsubstituted saturated heterocyclyl group.
H. The compound of any one of Paragraphs A-G, wherein when X³ is —C(R⁸)═C(R⁹)—, then R³ is not trifluoromethyl and is not alkoxy.
I. The compound of any one of Paragraphs A-H, wherein the compound is a compound according to Formula III

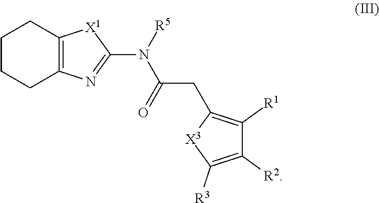

J. The compound of any one of Paragraphs A-I, wherein X³ is S or O.
K. The compound of any one of Paragraphs A-J, wherein X¹ is S.
L. The compound of any one of Paragraphs A-K, wherein when X¹ is S; X² is CH; X³ is S; R², R³, R⁴ and R⁵ are each independently H; and R⁶ and R⁷ taken together form a unsubstituted cyclohexyl group; then R¹ is not H.
M. The compound of any one of Paragraphs A-L, wherein when X¹ is S; X² is CH; X³ is S; R¹, R², R³, and R⁴ are each independently H; and R⁶ and R⁷ taken together form a unsubstituted cyclohexyl group; then R⁵ is not phenyl.
N. The compound of any one of Paragraphs A-M, wherein when X³ is S, then R¹, R², and R³ are each not trifluoromethyl.
O. A composition comprising a compound of any one of Paragraphs A-N and a pharmaceutically acceptable carrier.
P. A pharmaceutical composition for treating a viral infection, the composition comprising an effective amount of the compound of any one of Paragraphs A-N for treating a viral infection, and a pharmaceutically acceptable carrier, the viral infection comprising a Flaviviridae virus, Filoviridae virus, Retroviridae virus, Arenaviridae virus, Coronaviridae virus, Orthomyxoviridae virus, Paramyxoviridae virus, Togaviridae virus, or Rhabdovidae virus.
Q. The pharmaceutical composition of Paragraph P, wherein the viral infection comprises a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus.
R. The pharmaceutical composition of Paragraph P or Paragraph Q, wherein the effective compound selectively treats the viral infection.
S. The pharmaceutical composition of any one of Paragraphs P-R, wherein the pharmaceutical composition is packaged in unit dosage form.
T. The pharmaceutical composition of Paragraph S, wherein the unit dosage form is effective in preventing infection by, reducing symptoms associated with, reducing risk of transmission, or any combination of two or more thereof, of an virus when administered to a subject in need thereof, preferably wherein the virus comprises a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus, and/or preferably where the subject is human.
U. A method comprising:
   administering an antiviral effective amount of a compound of any one of Paragraphs A-N to a subject suffering from a viral infection;
   wherein the viral infection comprises a Flaviviridae virus, Filoviridae virus, Retroviridae virus, Arenaviridae virus, Coronaviridae virus, Orthomyxoviridae virus, Paramyxoviridae virus, Togaviridae virus, or Rhabdovidae virus.
V. The method of Paragraph U, wherein the viral infection comprises a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus.
W. The method of Paragraph U or Paragraph V, wherein the compound selectively treats the viral infection.
X. The method of any one of Paragraphs U-W, wherein the risk of infection by and/or transmission of a virus by said subject is decreased.
Y. The method of any one of Paragraphs U-X, wherein the administration comprises oral administration, parenteral administration, or nasal administration.
Z. The method of any one of Paragraphs U-Y, wherein the administration comprises subcutaneous injections, intravenous injections, intraperitoneal injections, or intramuscular injections.
AA. The method of any one of Paragraphs U-Z, wherein the administration comprises oral administration.
BB. The method of any one of Paragraphs U-AA, wherein the subject is human.

Other embodiments are set forth in the following claims, along with the full scope of equivalents to which such claims are entitled.

What is claimed is:

1. A compound according to Formula I 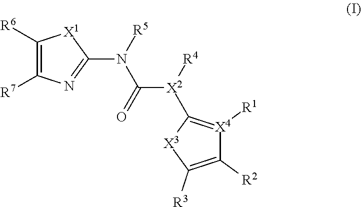
or a solvate and/or pharmaceutically acceptable salt thereof; wherein
X¹ is NH, S, or O;
X² is N, CH, or C-(unsubstituted alkyl);
X³ is S, O, or —C(R⁸)═C(R⁹)—;
X⁴ is N or C;
R¹, R², R³, R⁸ and R⁹ are each independently H, halo, cyano, trifluoromethyl, nitro, pentafluorosulfanyl, or a substituted or unsubstituted alkyl, alkoxy, aryl, aryloxy, alkynyl, cycloalkyl, heterocyclylalkyl, alkanoyl, alkanoyloxy, aryloyl, aryloyloxy, carboxylate, or ester group; provided that when X⁴ is N, then R¹ is absent;
R⁴ is H or unsubstituted alkyl;
R⁵ is H, or a substituted or unsubstituted alkyl, alkoxy, aryl, heteroaryl, alkynyl, cycloalkyl, heterocyclyl, heterocyclylalkyl, alkanoyl, or aryloyl group; or
R⁴ and R⁵ taken together are a substituted or unsubstituted C₂-C₄ alkylenyl group;
R⁶ and R⁷ are each independently H or unsubstituted alkyl, or R⁶ and R⁷ taken together form a substituted or unsubstituted aryl, or R⁶ and R⁷ taken together are a substituted or unsubstituted C₃-C₅ alkylenyl or —(CH₂)ₓ—O—(CH₂)_{y}— group, where x and y are each independently 1, 2, or 3 provided that when x is 3, y is 1, and when y is 3, x is 1.

2. The compound of claim 1, wherein X² is N or CH.

3. The compound of claim 1, wherein R⁴ is H, or R⁴ and R⁵ taken together are a substituted or unsubstituted C₂-C₄ alkylenyl group.

4. The compound of claim 1, wherein R⁶ and R⁷ taken together form a substituted or unsubstituted aryl or R⁶ and R⁷ taken together are a substituted or unsubstituted C₃-C₅ alkylenyl.

5. The compound of claim 1, where the compound is according to Formula II 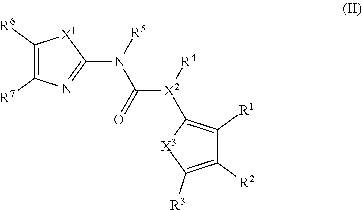
wherein
X² is N or CH;
R⁴ is H; or R⁴ and R⁵ taken together are a substituted or unsubstituted C₂-C₄ alkylenyl group;
R⁶ and R⁷ taken together form a substituted or unsubstituted aryl or R⁶ and R⁷ taken together are a substituted or unsubstituted C₃-C₅ alkylenyl.

6. The compound of claim 1, wherein R⁵ is a substituted or unsubstituted alkyl, a substituted or unsubstituted phenyl, or an unsubstituted heterocyclyl group.

7. The compound of claim 1, wherein R⁵ is a substituted or unsubstituted alkyl, a substituted or unsubstituted phenyl, or an unsubstituted saturated heterocyclyl group.

8. The compound of claim 1, wherein when X³ is —C(R⁸)═C(R⁹)—, then R³ is not trifluoromethyl and is not alkoxy.

9. The compound of claim 1, wherein the compound is a compound according to Formula III

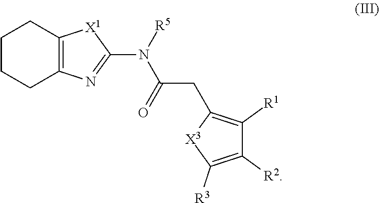

10. The compound of claim 9, wherein X³ is S or O.

11. The compound of claim 9, wherein X¹ is S.

12. The compound of claim 1, wherein when X¹ is S; X² is CH; X³ is S; R², R³, R⁴ and R⁵ are each independently H; and R⁶ and R⁷ taken together form a unsubstituted cyclohexyl group; then R¹ is not H.

13. The compound of claim 1, wherein when X¹ is S; X² is CH; X³ is S; R¹, R², R³, and R⁴ are each independently H; and R⁶ and R⁷ taken together form a unsubstituted cyclohexyl group; then R⁵ is not phenyl.

14. The compound of claim 1, wherein when X³ is S, then R¹, R², and R³ are each not trifluoromethyl.

15. A composition comprising a compound of claim 1 and a pharmaceutically acceptable carrier.

16. A pharmaceutical composition for treating a viral infection, the composition comprising an effective amount of the compound of claim 1 for treating a viral infection, and a pharmaceutically acceptable carrier, the viral infection comprising a Flaviviridae virus, Filoviridae virus, Retroviridae virus, Arenaviridae virus, Coronaviridae virus, Orthomyxoviridae virus, Paramyxoviridae virus, Togaviridae virus, or Rhabdovidae virus.

17. The pharmaceutical composition of claim 16, wherein the viral infection comprises a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus.

18-21. (canceled)

22. A method comprising:
administering an antiviral effective amount of a compound of claim 1 to a subject suffering from a viral infection;
wherein the viral infection comprises a Flaviviridae virus, Filoviridae virus, Retroviridae virus, Arenaviridae virus, Coronaviridae virus, Orthomyxoviridae virus, Paramyxoviridae virus, Togaviridae virus, or Rhabdovidae virus.

23. The method of claim 22, wherein the viral infection comprises a Chikungunya virus, Zika virus, Vesicular stomatitis Indiana virus, lymphocytic choriomeningitis virus, yellow fever virus, Ebola virus, human immunodeficiency virus, influenza A virus, herpes simplex virus 1, herpes simplex virus 2, Japanese encephalitis virus, West Nile Virus, severe acute respiratory syndrome coronavirus, Venezuelan equine encephalitis virus, or a respiratory syncytial virus.

24-25. (canceled)

26. The method of claim 22, wherein the administration comprises oral administration, parenteral administration, or nasal administration.

27-29. (canceled)
